(19) United States
(12) Patent Application Publication
(10) Pub. No.: US 2016/0296208 A1
(43) Pub. Date: Oct. 13, 2016

(54) Intravascular Photoacoustic and Ultrasound Echo Imaging

(71) Applicant: Board of Regents, The University of Texas System, Austin, TX (US)

(72) Inventors: Shriram Sethuraman, Briarcliff Manor, NY (US); Stanislav Y. Emelianov, Austin, TX (US); Richard W. Smalling, Houston, TX (US); Salavat R. Aglyamov, Austin, TX (US)

(21) Appl. No.: 14/995,802

(22) Filed: Jan. 14, 2016

Related U.S. Application Data
(63) Continuation of application No. 12/449,384, filed on Oct. 13, 2010, now abandoned.
(60) Provisional application No. 60/900,506, filed on Feb. 9, 2007.

(51) Int. Cl. A61B 8/08; A61B 8/00; A61B 8/12; A61B 5/02; A61B 8/14; A61B 5/00

(52) U.S. Cl. CPC A61B 8/5261; A61B 8/14; A61B 8/5207; A61B 5/0095; A61B 8/12; A61B 5/6852; A61B 5/02007; A61B 8/445; A61B 8/0891; A61B 8/4483; A61B 5/0075

(57) ABSTRACT

The invention relates to photoacoustic imaging and ultrasound echo imaging, in combination, and applies in particular to the field of imaging a lumen of an organ or vessel of a subject, wherein the images are acquired from within a lumen of the organ or vessel, especially a lumen of a blood vessel to diagnose and treat vascular disease. An exemplary embodiment of the invention is a catheter having an ultrasound transducer, the transducer comprising a probe suitable for generating and detecting photoacoustic signals and ultrasound echo signals, wherein the photoacoustic signals and the ultrasound echo signals are convertible to images which are integrated into an enriched image. The photoacoustic signals are generated by a multiplicity of energy sources suitable for inducing the walls of the blood vessel to generate acoustic waves, wherein the energy sources are arrayed in an annulus around the flexible tubular member.

### CROSS-REFERENCE TO RELATED APPLICATIONS

This application is a Continuation of U.S. patent application Ser. No. 12/449,384 filed on Oct. 13, 2010, which is a 35 U.S.C. 371 National Stage application of International Application PCT/US08/01379, filed Feb. 1, 2008, which claims the benefit of U.S. Provisional Application No. 60/900,506, filed Feb. 9, 2007. The contents of each are incorporated by reference in their entirety.

### FIELD

The invention relates generally to photoacoustic imaging and ultrasound echo imaging in combination, and applies in particular to the field of imaging the walls that define a lumen of an organ or vessel of a subject, wherein the images are acquired from a vantage point within a lumen of the organ or vessel, especially a lumen of a blood vessel to diagnose and treat vascular disease.

### BACKGROUND

Cardiovascular disease (“CVD”) is the principal cause of mortality in the United States. The complications associated with CVD are primarily caused by atherosclerosis—a disease of the arteries. High levels of plasma low density lipoprotein cholesterol lead to the accumulation of lipids and to the formation of plaques deposited in the walls of the arteries (Ross, R., “The pathogenesis of atherosclerosis: a perspective for the 1990's,” Nature 362: 801-809, 1993). Plaque formation is further thought to be accompanied by an inflammatory response with the recruitment of monocyte-derived macrophages. X-ray angiography is used clinically to detect plaque formations and to evaluate their impact on narrowing and ultimately obstructing the arterial lumen.

Advances in the biology of the disease and its progression have brought to light the existence of so-called “vulnerable” plaques (Naghavi, P. et al., “From vulnerable plaque to vulnerable patient: a call for new definitions and risk assessment strategies: Part I,” Circulation 108: 1664-1672, 2003; Kolodgie, F. D. et al., “The thin-cap fibroatheroma: a type of vulnerable plaque: the major precursor lesion to acute coronary syndromes, Curr. Opin. Cardiol. 16: 285-292, 2002; Stary, H. C., et al., “A definition of advanced types of atherosclerotic lesions and a histological classification of atherosclerosis. A report from the Committee on Vascular Lesions of the Council of Arteriosclerosis, American Heart Association,” Arterioscler. Thromb. Vase. Biol. 15: 1512-1531, 1995): Morphologically (Virmani, R., et al., “Lessons from sudden coronary death: a comprehensive morphological classification scheme for atherosclerotic lesions,” Arterioscler. Thromb. Vase. Biol. 20:1262-1275, 2000) and compositionally, vulnerable plaques (that is, a plaque that acquires the tendency to rupture) cover a spectrum of types. Other structural and functional characteristics of vulnerable lesions have been identified, among them, vascular remodeling, vasa vasorum neovascularization and formation of intra-plaque hemorrhage (Glagov, S., et al., “Compensatory enlargement of human atherosclerotic coronary arteries,” N. Engl. J. Med. 216: 1371-1375, 1987). In general, each type has its own pathological significance but, typically, either myocardial infarction or stroke follows upon the rupture of a plaque.

Plaques may comprise connective tissue extracellular matrix (including, without limitation, collagen, proteoglycans and fibronectin), cholesterol, calcium, blood, monocyte-derived macrophages and smooth muscle cells (Naghavi et al., op. cit.). Different proportions of the above-mentioned components may give rise to a heterogeneity or spectrum of lesions. The components primarily affect the innermost, arterial layer (the “intima,” or layer that generally defines the lumen of the blood vessel). Secondary lesions may also infiltrate the outer layers (“media” and “adventitia”) of the arterial wall. A widely accepted model of an atherosclerotic lesion comprises a thin fibrous cap (approximately 60-150 micrometers) overlying a large, lipid-filled core (Kolodgie, F. D. et al., op. cit.). As lipids and macrophages accumulate in the lesion, its fibrous cap tends to rupture as part of an inflammatory process. Atherosclerosis, therefore, is an inflammatory disease with a series of highly specific cellular and molecular responses (Libby et al., “Inflammation and atherosclerosis,” Circulation 105: 1135-1143, 2002; Shah, P. K., “Mechanisms of plaque vulnerability and rupture,” J. Am. Coll. Cardiol. 41: 158-228, 2003). Apart from the most common type of plaques comprised of lipids and macrophages, the rupture-prone plaques may also contain calcium, blood, collagen and smooth muscle cells (Naghavi, M. et al. op. cit.). Therefore, the heterogeneous composition of the plaque is a major factor in deciding appropriate therapy.

The ability to assess the vulnerability of plaque formations has sufficient clinical value to have motivated a number of efforts to image and distinguish rupture-prone plaque from less ominous lesions (Fayad, Z. A. et al., “Clinical imaging of the high-risk or vulnerable atherosclerotic plaque,” Circ. Res. 89: 305-316, 2001). Magnetic resonance imaging (“MRI”), despite the time and expense it entails, and its marginal resolution, has the advantage of being non-invasive. Electron-beam computed tomography (“EBCT”), specific for calcium-based plaque, awaits further research to determine its applicability to vulnerable plaque. Optical coherence tomography (“OCT”) is a high resolution technique in principle but, in practice, the light-scattering inherent in it compromises image quality (Fujimoto, J. G. et al., “High resolution in vivo intra-arterial imaging with optical coherence tomography.” Heart 82: 128-133, 1999). Inasmuch as the temperature of a plaque tends to rise as macrophage activity within it increases, thermographic modalities may eventually prove useful. Finally, intravascular ultrasound echo imaging (“IVUS”), (Nissen, S. E. et al., “Intravascular ultrasound: novel pathophysiological insights and current clinical applications,” Circulation 103: 604-616, 2001), a well-developed technology widely used in cardiac catheterization, is coming into service to identify vulnerable plaque. Palpography is an IVUS modality that distinguishes among types of plaque on the basis of a plaque's specific deformability under the force of arterial pulse pressure. Another IVUS modality measures the “echogenicity” of the arterial wall by analyzing particular details of the echoes that provide the raw data for conventional ultrasound imaging. Low echogenicity correlates with vulnerable (soft, lipid rich) plaque.

The most common manifestation of the disease is a progressive constriction of the blood vessels affecting blood flow. Generally, the structural change caused by luminal stenosis is observed through angiographic images of the artery and has been a standard diagnostic indicator of the disease. However, the ability of X-ray angiography to detect vulnerable plaques is minimal Ambrose, J. A. et al., “Angiographic progression of coronary artery disease and development of myocardial infarction,” J Am. Coll. Cardiol. 12: 56-62, 1998; Little, W. C. et al., “Can coronary angiography predict the site of a subsequent myocardial infarction in patients with mild-to-moderate coronary artery disease?” Circulation 78: 1157-1166, 1988). Several other imaging techniques such as optical coherence tomography (OCT), magnetic resonance imaging (MRI), ultrafast computed tomography, thermography, intravascular palpography, angioscopy and raman spectroscopy are under investigation but have limitations and are not yet clinically available (Fayad, Z. A. et al., op. cit.). Although intravascular ultrasound (IVUS) is clinically available, the technique needs improvement in the detection of vulnerable plaques.

### SUMMARY

The invention relates generally to photoacoustic imaging and ultrasound echo imaging in combination. The invention enables the artisan to combine photoacoustic and ultrasound echo images acquired from vantage points within the lumen of an organ or vessel of a subject, especially images of the walls of a blood vessel. The combination of intravascular photoacoustic (“IVPN”) imaging and intravascular ultrasound (“IVUS”) imaging in effect superimposes IVPA technology on conventional IVUS technology to solve existing medical needs.

A variety of embodiments is contemplated for the present invention. The invention may, for example, be embodied in a device comprising an optical excitation probe, an ultrasonic hydrophone probe and an ultrasound generating probe, wherein the probes are sized to fit into a lumen of an organ of a subject. The organ may be a blood vessel. In some embodiments, the ultrasonic hydrophone probe is combined with the optical excitation probe in such manner as to comprise a photoacoustic imaging probe. Similarly, the hydrophone probe may be combined with the ultrasound generating probe in such manner as to comprise an ultrasound transducer probe. Generally, the ultrasound transducer probe is capable of acquiring an ultrasound echo image of an object and the photoacoustic imaging probe is capable of acquiring a photoacoustic image of the object. Preferably, the ultrasound echo image and the photoacoustic image can be co-registered.

Catheters that embody the invention are sized to fit into a lumen of an organ of a subject. The organ may be a blood vessel.

In one catheter embodiment, the catheter comprises:
a) an elongated flexible tubular member having
   (i) a longitudinal axis and proximal and distal ends,
   (ii) a first lumen extending longitudinally there through, said first lumen sized to receive a guide wire,
   (iii) a second lumen extending longitudinally there through, said second lumen sized to accommodate an electrically conductive wire, and
   (iv) an ultrasound transducer disposed at the distal end of the flexible tubular member, the transducer comprising a probe suitable for generating and for detecting photoacoustic signals and ultrasound echo signals, wherein the photoacoustic signals and the ultrasound echo signals are convertible to images, wherein the images are integrated into an enriched image,
b) a multiplicity of energy sources suitable for inducing the walls of the body vessel to generate acoustic waves, wherein the energy sources are arrayed in an annulus around the flexible tubular member and disposed to direct energy onto a wall segment of the-body vessel, and
c) an outer sheath surrounding the flexible tubular member, the flexible tubular element further comprising a drug delivery element suitable for delivering therapeutic agents to the body vessel.

In one catheter embodiment, the catheter comprises:
a) a tubular member suitable for insertion into a vessel in the body of a patient, the tubular member having
   (i) a longitudinal axis and proximal and distal ends,
   (ii) a first lumen extending longitudinally there through, said first lumen sized to receive a guide wire,
   (iii) a second lumen extending longitudinally there through, said second lumen sized to accommodate an electrically conductive wire, and
b) an ultrasound transducer disposed at the distal end of the flexible tubular member, the transducer comprising means for generating and for detecting photoacoustic signals and ultrasound echo signals.

In one catheter embodiment, the catheter comprises:
a) a tubular member suitable for insertion into a vessel in the body of a patient, the tubular member having a longitudinal axis, and proximal and distal ends, and
b) an ultrasound transducer disposed at the distal end of the flexible tubular member, the transducer comprising means for generating and for detecting photoacoustic signals and ultrasound echo signals.

The present invention may also be embodied in a variety of systems. One such system comprises:
a) a photoacoustic catheter sized to fit within a lumen of an organ of a subject, the photoacoustic catheter having a photoacoustic probe comprising an optical excitation probe, an ultrasonic hydrophone probe, and indicia for identifying a locus of the photoacoustic probe in the lumen,
b) an ultrasound echo catheter sized to fit within that lumen, the ultrasound echo catheter having an ultrasound transducer probe, and indicia for identifying a locus of the ultrasound transducer probe in the lumen,
c) a light source interfaced with the optical excitation probe of the photoacoustic catheter, and
d) a pulser/receiver in communication with the light source and the ultrasonic hydrophone probe of the photoacoustic catheter.

Preferably, the photoacoustic probe of the photoacoustic catheter, the transducer probe of the ultrasound echo catheter and the pulser/receiver are controlled by a microprocessor. The light source is preferably a laser.

In one embodiment of the invention, the photoacoustic catheter and the ultrasound echo catheter of the aforementioned system are combined within a single sheath to comprise a combination catheter sized to fit into a lumen of an organ of a subject.

A variety of methods may also embody the invention. One of these is a method of mapping and identifying plaque in a blood vessel comprising the steps of:
a) providing a blood vessel suspected of having plaque disposed therein,
b) feeding a catheter comprising a photoacoustic imaging probe and an ultrasound transducer probe into a lumen of said blood vessel,
c) acquiring an ultrasound echo image and a photoacoustic image of an element of a wall segment of the blood vessel, and
d) repeating step (c) until an ultrasound echo image and a photoacoustic image of the wall segment are acquired.

In one embodiment, the data on which the images are based is stored for later processing. In one embodiment the data is processed in real-time. Preferably, the acquired images of the wall segment are mapped onto the blood vessel, preferably as superimposed images. Generally, the photoacoustic image is acquired repeatedly over a range of wavelengths of laser light. It is also contemplated that, generally, a plurality of contiguous wall segments are imaged and mapped according to the method.

### BRIEF DESCRIPTION OF THE DRAWINGS

FIG. 1A schematically depicts an experimental set up for combining IVPA and IVUS imaging according to an embodiment of the present disclosure

FIG. 1B schematically depicts a block diagram of a combined IVUS/IVPA imaging system according to an embodiment of the present disclosure.

FIG. 2 graphically illustrates the experimental setup shown in FIG. 1A.

FIG. 3 depicts a representative A-line containing IVPA and IVUS signals from a phantom with inclusions, wherein a four microsecond (4 μs) delay was used to separate the IVUS pulse-echo signal following the IVPA signal.

FIG. 4 depicts a flow diagram of the control algorithm for image acquisition according to an embodiment of the present disclosure.

FIG. 5A is a cross-sectional image of a phantom with two inclusions, wherein IVUS (20 MHz) was used for image acquisition.

FIG. 5B is a cross-sectional image of a phantom with two inclusions, wherein IVPA (20 MHz) was used for image acquisition.

FIG. 5C is a cross-sectional image of a phantom with two inclusions, wherein IVUS/IVPA (20 MHz) was used for image acquisition.

FIG. 5D is a cross-sectional image of a phantom with two inclusions, wherein IVUS (30 MHz) was used for image acquisition.

FIG. 5E is a cross-sectional image of a phantom with two inclusions, wherein IVPA (30 MHz) was used for image acquisition.

FIG. 5F is a cross-sectional image of a phantom with two inclusions, wherein IVUS/IVPA (30 MHz) was used for image acquisition.

FIG. 5G is a cross-sectional image of a phantom with two inclusions, wherein IVUS (40 MHz) was used for image acquisition.

FIG. 5H is a cross-sectional image of a phantom with two inclusions, wherein IVPA (40 MHz) was used for image acquisition.

FIG. 5I is a cross-sectional image of a phantom with two inclusions, wherein IVUS/IVPA (40 MHz) was used for image acquisition.

FIG. 6A is a cross-sectional image of an excised sample of a rabbit artery having a field of view diameter of 6.75 millimeters (mm), wherein IVUS was used for image acquisition.

FIG. 6B is a cross-sectional image of an excised sample of a rabbit artery having a field of view diameter of 6.75 millimeters (mm), wherein IVPA (532 nanometer excitation wavelength, 40 MHz IVUS imaging catheter) was used for image acquisition.

FIG. 6C is a cross-sectional image of an excised sample of a rabbit artery having a field of view diameter of 6.75 millimeters (mm), wherein IVUS/IVPA (20 MHz) was used for image acquisition.

FIG. 7A graphically illustrates a forward imaging mode configuration representative of the type used for ex vivo IVPA imaging experiments disclosed herein.

FIG. 7B is an image of an intact rabbit aorta with an IVUS catheter inserted in the lumen.

FIG. 7C graphically illustrates a backward imaging mode with ultrasound transducer and light delivery system positioned on the same side.

FIG. 7D is an image of a sample carotid artery opened and imaged with the intima facing the imaging probe.

FIG. 8A is an image from an IVUS B-scan of a atherosclerotic aorta with plaque creating a decreased dimeter of the lumen, the image having a 9 mm diameter field of view and 1 mm radial marks.

FIG. 8B is an IVPA image of an aorta showing the photoacoustic response from the aorta and plaque, wherein the hypoechoic region in the image at 7 o'clock to 9 o'clock outlines suspected lipid formation.

FIG. 8C is an IVUS image of a control aorta excised from a normal rabbit, wherein the photoacoustic response from the fibrous components of the aortic wall is nearly homogeneous.

FIG. 8D is an IVPA image of a control aorta excised from a normal rabbit, wherein the photoacoustic response from the fibrous components of the aortic wall is nearly homogeneous.

FIG. 9A is a Hemotoxylin and Eosin (H&E) stained histology image of an aorta from an atherosclerotic rabbit.

FIG. 9B is a Picrosirius red stained histology image of an aorta from an atherosclerotic rabbit.

FIG. 9C is a RAM-11 stained histology image of an aorta from an atherosclerotic rabbit.

FIG. 9D a Hemotoxylin and Eosin (H&E) stained histology image of an aorta from a control rabbit.

FIG. 9E is a Picrosirius red stained histology image of an aorta from a control rabbit.

FIG. 9F is a RAM-11 stained histology image of an aorta from a control rabbit.

FIG. 10A is an ultrasound echo B-scan image (acquired in the backward imaging configuration and measuring 15 mm laterally and 4.6 mm in depth) of a carotid artery.

FIG. 10B is a photoacoustic image of the carotid artery in FIG. 10A.

FIG. 11A is a ultrasound echo image (6.4 mm by 2.1 mm) of a carotid artery with plaque immersed in saline.

FIG. 11B is a photoacoustic image (6.4 mm by 2.1 mm) of a carotid artery with plaque immersed in saline.

FIG. 11C is an ultrasound echo image (6.4 mm by 2.1 mm) of a carotid artery with plaque immersed in blood.

FIG. 11D is a photoacoustic image (6.4 mm by 2.1 mm) of a carotid artery with plaque immersed in blood.

FIG. 12A graphically depicts one side view and two cross section views (at positions “A” and “B”) of an integrated IVUS/IVPA catheter, wherein the catheter has a single element ultrasound transducer.

FIG. 12B graphically depicts one side view and two cross section views (at positions “A” and “B”) of an integrated IVUS/IVPA catheter, wherein the catheter has a transducer array.

FIG. 13A is an array of cross-sectional combined images (at nine different wavelengths) of a atherosclerotic rabbit artery.

FIG. 13B is an array of cross-sectional combined images (at nine different wavelengths) of a normal rabbit artery.

FIG. 14A is a derivative image derived using the images of the atherosclerotic aorta in FIG. 13A.

FIG. 14B is a derivative image derived using the images of the normal aorta in FIG. 13B.

FIG. 15A is a temperature image of an aorta exposed to energy sufficient for photoacoustic images at 0.1 milliseconds (ms).

FIG. 15B is a temperature image of an aorta exposed to energy sufficient for photoacoustic images at 5 milliseconds (ms).

FIG. 15C is a temperature image of an aorta exposed to energy sufficient for photoacoustic images at 10 milliseconds (ms).

FIG. 15D is a temperature image of an aorta exposed to energy sufficient for photoacoustic images at 15 milliseconds (ms).

### DETAILED DESCRIPTION

The invention enables the practitioner to acquire an image of a tissue or tissue element of an organ or vessel of a subject. The image is acquired from a vantage point within a lumen of the organ or vessel. The acquired image contains morphological information derived from ultrasound echo interrogation and functional information derived from photoacoustic ultrasound interrogation of the tissue. In particular, the invention enables the practitioner, by means of an intravascular catheter, to “map” (that is, to identify the position of a point in space relative to a reference point) plaque formations in the wall of a blood vessel, and to distinguish vulnerable plaques therein.

Biological tissues have photoelastic properties. That is, when light impinges on a tissue, the light's energy, as the tissue absorbs it, elastically deforms the tissue. It is thought that a beam of light, “chopped” at an appropriate frequency, drives a thermal deformation-relaxation cycle in the tissue that, in turn, creates sound waves. When such waves emanate from the affected tissue at ultrasonic frequencies, an ultrasonic detector can detect them. These light-induced ultrasonic waves, furthermore, can be converted into images reflective of the structure and, especially, the composition of the tissue.

Laser-induced photoacoustic tomography (“PAT”) is such an imaging modality. It requires a source of laser energy and a means of detecting ultrasonic waves, but it avoids the problem of light scattering that limits resolution in optical imaging. Moreover, it is not vulnerable to the contrast and speckle disadvantages of conventional ultrasound imaging (“ultrasound echo imaging”), and does not involve ionizing radiation.

Conventional ultrasound imaging, which relies entirely on sound waves generated by an ultrasound generator and received back as “echoes” reflected off of the tissue of interest, provides a qualitatively different image that has its own advantages.

Both imaging modalities have assumed roles in the diagnosis and treatment of diseases of the cardiovascular system.

The term “intravascular” as used herein refers to a site within a blood vessel. The referenced site may be within a lumen of the vessel or within the wall of the vessel, as the context so admits. Generally herein, the vessel or blood vessel is an artery but the term encompasses any vessel comprising the cardiovascular system of a human or animal.

The term “organ” herein encompasses any structure in a subject (including humans, animals and vegetative systems) that has a lumen capable of accommodating a photoacoustic probe and an ultrasound transducer probe. The term encompasses blood vessels and, by way of example and not limitation, such passages as the lymphatic vessels, the esophagus, stomach, intestine, ureter, urethra, trachea, sinuses, Eustachian tubes, etc., and ducts including without limitation bile ducts, pancreatic ducts.

“Lumen” as used herein refers to a passageway or bore extending into or through an organ or a segment thereof and defined by the tissue of the organ that comprises the walls that surround the lumen. Such lumen may be virtual (that is, not an actual open space) or even constructed, as by a surgical procedure.

In certain embodiments, the instant invention employs an IVUS probe. In IVUS imaging, an IVUS catheter is advanced on a guide wire 40 through an access catheter 90 to the distal part of the artery under examination. The distal end-region of the IVUS catheter is adapted to emit an ultrasound beam in a particular direction and to receive the beam back as backscatter. While applicants will not be bound by any theory of the mechanisms underlying embodiments of their invention, it is generally believed that the time between transmission of the ultrasound pulse or pressure wave and reception of the reflected or backscattered wave or echo is directly related to the distance between the source and the reflector, the reflector in this case being a tissue element. To form a transverse cross-sectional image of the vessel in real-time, the ultrasound beam is rotated at several revolutions per second. A preferred rate is 30 revolutions per second (that is, 30 images per second). The iSight™ intravascular ultrasound echo catheter (Boston Scientific, Natick, Mass.), which has a mechanically scanned single element transducer 150, may be employed. In another embodiment, a catheter having an array of electronically scanned transducers, such as the Avanar® FIX intravascular ultrasound echo imaging catheter 275 (Volcano Corporation, Rancho Cordova, Calif.), may be used.

As used herein, the term “probe,” whether applied to an ultrasound echo probe, a photoacoustic probe, an excitation probe or otherwise, refers to an element that serves a signal generating function or a signal reception function or both. Thus an “ultrasound probe” or “ultrasound transducer probe” or “ultrasound echo probe” refers to an element capable of sending ultrasonic waves (waves of a frequency or pitch higher than that to which the human ear is sensitive) or receiving such waves. The term “probe” encompasses accessory elements necessary for the probe to function in the several embodiments of the invention. For example, some of the “ultrasound transducer probes” identified herein, to be useful in the context of the invention, require a motor 45 to rotate the transducer element itself. To the extent required for relevant functionality, then, the motor would be considered part of the ultrasound transducer probe.

A “photoacoustic probe” or “photoacoustic ultrasound probe” refers to an element capable of emitting photons and receiving acoustic signals (i.e., “sound waves”). A probe, as used herein, need not be a self-contained physical entity: several physical elements may cooperate to generate the probe's function. A photoacoustic probe, for example, may comprise (a) a material such as a piezoelectric crystal which, by oscillating when driven by sound waves, generates an oscillating electric field and (b) in proximity to the oscillator, a different material such as a fiberoptic filament or fiber or a bundle of such fibers that can emit a beam of photons. The region of such fiberoptic filament from which the beam of photons emanates is a non-limiting example of an “optical excitation probe” as that term is used herein. In this example of an optical excitation probe, the probe receives its photons from a light source (preferably a coherent light source such as a laser) that interfaces with the photoacoustic probe. The term “interface” herein, is intended to convey a functional concept. That is, the laser and the photo acoustic probe need not be directly compatible: any of a number of methods and devices can be used to “interface” the two elements. One such element in this case is the fiberoptic bundle that carries photons emanating from the laser to the excitation probe.

The term “laser” as used herein refers to any device capable of generating a beam of coherent light, and “laser light” refers to any such beam.

Terms such as “ultrasound echo probe,” “ultrasound echo image,” and “ultrasound echo catheter” are employed herein principally to distinguish echo-based ultrasound technologies from photoacoustic-based technologies. The “echoes” of echo-based technologies have a range of properties and applications. Use of the word “echo” herein is not intended to limit the echo-based technologies that artisans may employ in practicing various embodiments of the invention. The terms “ultrasonic” and “ultrasound” are used interchangeably herein.

Other excitation probes are consistent with the invention. For example, it is not necessary that light be transported to the probe, whether by fiberoptic means or otherwise, to have an “optical excitation probe.” A laser diode or an array of laser diodes disposed in proximity to the aforementioned piezoelectric crystal oscillator and activated by electricity delivered by wire would be one alternative. Indeed, although it is most preferred to employ the energy of photons in the several embodiments of the invention, any source of energy that can induce tissue to generate the acoustic waves required to assay the optical characteristics of the tissue in accordance with the invention is within the scope of the invention.

Conveniently, the oscillator can serve multiple functions in some embodiments of the invention. Typical ultrasonic transducers convert the mechanical energy of sound waves into electrical energy that can be readily employed as information with which to construct images of objects. This is the “microphone” function of ultrasound transducers, for sound waves in air, or the “hydrophone” function for sound waves in liquids. In some embodiments of the invention, both photoacoustic probes and ultrasound echo probes utilize the hydrophone function. Ultrasound transducers also convert electrical energy into the mechanical energy of sound waves, the reflection of which from a relatively non-compliant surface of an object become the “echoes” that give rise to ultrasonic images of the object. In preferred embodiments, one selects an ultrasound transducer whose dynamic range permits the transducer to be responsive to both the photoacoustic waves of interest and to the ultrasound echoes of interest.

As used herein, the phrase “in combination” refers to two or more devices made capable of functioning cooperatively by being combined. By way of pertinent example, some embodiments of the invention are capable of superimposing a photoacoustic image upon an ultrasound echo image (the images are said to be “co-registered”) because, in the embodiment, a photoacoustic probe is combined in fixed relation to an ultrasound echo probe. Notwithstanding the foregoing, the invention also applies to embodiments where the configuration of the photoacoustic probe and the ultrasound transducer do not directly result in co-registration. That is, embodiments are contemplated wherein a photoacoustic probe acquires a pre-determined registration mark and a separate ultrasound transducer acquires the same registration mark, thus permitting the photoacoustic data and the echo data to be co-registered. Such registration marks may be referred to herein as “indicia.” Indicia are used for co-registration and for mapping a particular image (of, say, a plaque formation) to a particular locus within a vessel.

As used herein, “object” refers to any physical entity, regardless of its size, shape, composition or position in space, which is tangible in the sense of being directly or indirectly within the grasp of the senses. An “image” refers to a likeness of an object or an attribute of an object such as size, shape, color, composition or position in space.

A typical IVUS image distinguishes three layers (intima, media and adventitia) disposed annularly about the lumen of the artery being imaged. The intima, normally appearing as a thin layer of endothelial cells, substantially and often unevenly thickens in atherosclerosis. From the IVUS data, one estimates vessel area based on measurements of the media-adventitia border. Plaque area is derived by subtracting luminal area from vessel area.

IVUS images readily reveal calcified plaques. Other lesions also appear but are not generally distinguishable as to type (Franzen, D. et al., “Comparison of angioscopic, intravascular ultrasonic, and angiographic detection of thrombus in coronary stenosis,” Am. J Cardiol. 82: 1273-1275, A9, 1998). By pulling the IVUS catheter back through the vessel slowly (preferably <1 mm/sec), serial images can be acquired. Collectively, these images comprise a map of lesion sites in the vessel.

The invention may be embodied in a device that combines the modalities of ultrasound echo imaging and spectroscopic photoacoustic imaging in a configuration suitable for placement within, and movement along, the lumen of a blood vessel ex vivo or in vivo. An example of such an embodiment is a catheter having at its distal end-region an ultrasound echo imaging probe and an excitation energy probe or “optical excitation probe.” The excitation probe is disposed in relation to the ultrasound echo probe such that the two can cooperate to function as a photoacoustic imaging probe. As used herein, the term “catheter” refers to any elongate structure that is capable of being “fed,” “threaded” or “snaked” into and along the lumen of a tubular structure. As such, materials suitable for catheters are generally flexible but afford the catheter sufficient resilience in axial tension to accommodate axial forces (“pushing” and “pulling”). The term “sized” is repeatedly used herein to help characterize the probes and catheters that embody the invention. In “sizing” a device for insertion into a lumen of an organ or vessel, the artisan will understand that the smallest size of a probe or catheter will be dictated mainly by the limits of whatever miniaturization technology can at any time be applied to the elements that must be combined to make the device effective. The maximum size will be dictated mainly by the extent to which the device can safely distend the lumen of interest.

The invention may also be embodied in a method for identifying and mapping the locations of plaque in a blood vessel. In this embodiment, the blood vessel is examined with the devices and methods of the invention to acquire data on spectral variations in photon absorption by individual components of plaque formations embedded in or on the luminal aspect of a wall of the blood vessel. Methods that embody the invention use the acquired data to detect and map plaque, and to identify the types of plaque deposited in and on the walls of the blood vessel. While the applicants will not be bound by any theory of the mechanisms underlying embodiments of their invention, it is thought that a plaque formation made up predominantly of cholesterol, for example, will have different elastic properties than a plaque formation made up predominantly of calcium deposits. Even within a single plaque formation of a particular type (e.g., a “cholesterol plaque”), certain embodiments of the invention may reveal photoelastic heterogeneities having diagnostic implications.

In some embodiments, to enrich the “lesion map” with functional information that invests the lesions with a pathological identity to guide diagnosis and therapy, the invention integrates photoacoustic images into the IVUS images. Photoacoustic imaging is a relatively new technique aimed at providing functional information about tissues based upon differential absorption of photon energy by issue elements (Oraevsky, A. A, et al., op. cit.; Beard, P. C. et al., “Characterization of post mortem arterial tissue using time-resolved photoacoustic spectroscopy at 436, 461 and 532 nm,” Phys. Med. Biol. 42: 177-198, 1997; Hoelen, C. G. et al., “Detection of photoacoustic transients originating from microstructures in optically diffuse media such as biological tissue,” IEEE Trans Ultrasonic Ferroelectric Frequency Control 48: 37-47, 2001; Wang, X. et al., op. cit.). While applicants will not be bound by any theory of the mechanisms underlying embodiments of their invention, it is believed that the absorption measurements in photoacoustic imaging do not depend upon the reflection, scattering or refraction of light. Instead, the absorbed energy is thought to heat a region within the tissue element, causing the region to expand, thus stressing or “stretching” the immediately surrounding material. Provided the material can withstand the stress (i.e., the amount of energy absorbed is small enough to satisfy the so-called “stress confinement condition”), the result is a thermoelastic expansion. If the energy is applied for a sufficiently short time, the absorbed energy is thought to dissipate, whereupon the stretched tissue will contract. Not unlike a vibrating violin string, the cycles or waves of expansion and contraction are acoustic. In a high-frequency regime, the waves are ultrasonic and can be picked up by the ultrasound transducer resident in the IVUS catheter.

Just as the ultrasound data from ultrasonic echoes can be converted into images, so can ultrasound data from thermoelastic oscillators. The latter images, however, are thought to be “optical” in nature because the absorption of light by a tissue element is a function of the optical properties of that element. Arterial vessel walls comprise blood, collagen and proteoglycans, each of which has an unique light absorption spectrum or “color.” Thus, in a sense, photoacoustic imaging is a way of “hearing” colors. For example, volume-for volume, blood absorbs light of wavelength 400 nanometers 100 times more strongly than cells disposed on the wall of the aorta. Acoustic waves generated by light shone at that wavelength in a blood vessel are therefore probably coming from blood. At 700 nanometers, however, blood absorbs light much less intensely. Using a single element IVUS imaging catheter to acquire photoacoustic data (but not echo data), Sethuraman, S. et al. (“Intravascular photoacoustic imaging to detect and differentiate atherosclerotic plaques,” IEEE International Ultrasonics Symposium, Rotterdam, Netherlands 2005) were able to detect (but not map) plaque formations of different composition.

To apply photoacoustic imaging effectively to distinguish vulnerable plaque from other types when one encounters a plaque formation in an artery, it is preferable to be able not only to identify the type of plaque encountered but also to know where the particular plaque in question has infiltrated the structure of the vessel wall. For this, one should address the problem of putting the IVUS image into registration with the IVPA image so that one can acquire temporally consecutive (as close to simultaneous as practical), spatially concurrent ultrasound echo and photoacoustic signals. In some embodiments, this “co-ordinate control” is achieved in part by employing a “pulser/receiver.” Under the control of algorithms programmed into a microprocessor, the pulser/receiver, which is in electrical communication with the microprocessor, the ultrasound transducer probe and the control elements of the laser system interfaced with the photoacoustic probe, allows the user to control the optical excitation signal and the ultrasound echo signal temporally as a function of photoacoustic and echo signals received. In some embodiments, a co-registered image is acquired by applying excitation energy from outside the vessel at a pre-determined site in a segment of the vessel's wall, and echo-generating ultrasound from an IVUS probe on an IVUS catheter inside the vessel. A “wall segment” refers to a cross-sectional volume of a vessel wall, such cross-section having an arbitrary thickness, preferably not less than the resolution of the method. A variety of well-known methods can be used to record the location of the segment from which an image is being acquired, one of which is to note the depth of penetration of the IVUS catheter. An given ultrasound echo image is said to be “co-registered” with a given photoacoustic image when the latter can be specifically matched to the former by whatever means. In some embodiments, the configuration of the elements enforces co-registration. In others, mapping data are used to achieve co-registration or superimposition.

In a preferred embodiment, the IVUS catheter carries not only an IVUS probe but a plurality of IVPA probes that together illuminate (and penetrate) the entire wall of a segment of the vessel from inside the vessel. In a most preferred embodiment, the IVUS probe rotates as it sends and receives signals, thus acquiring image data through 360°. By imaging contiguous wall segments serially, an entire vessel can be imaged and reconstructed tomographically.

### Example 1

### Design of One Embodiment of a Combined IVUS/IVPA Imaging System

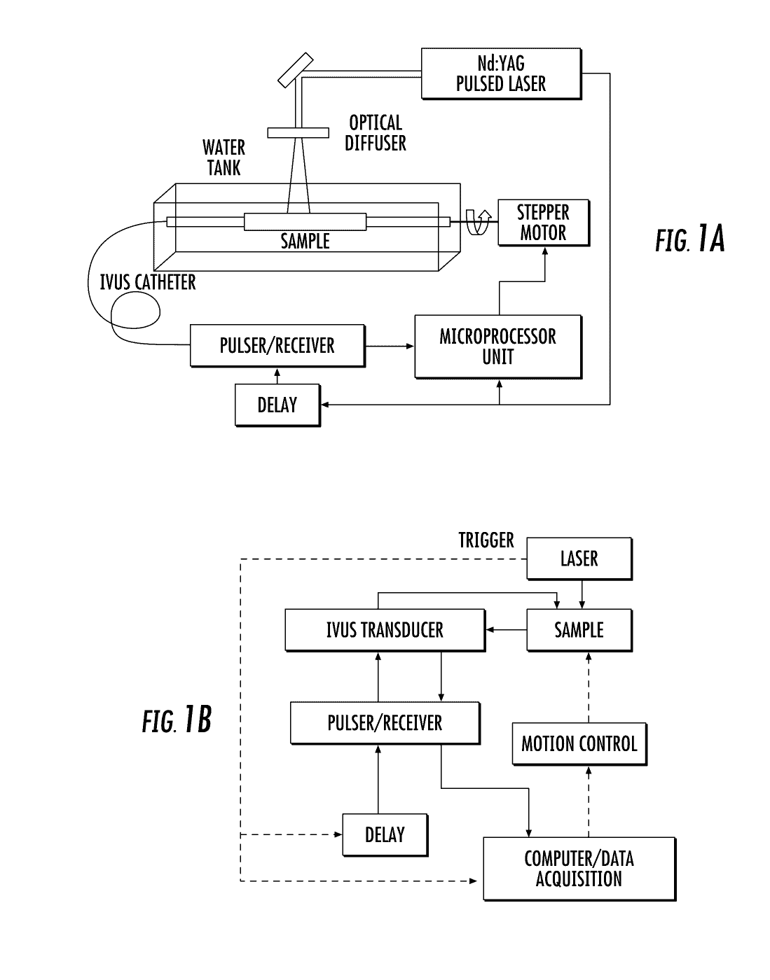

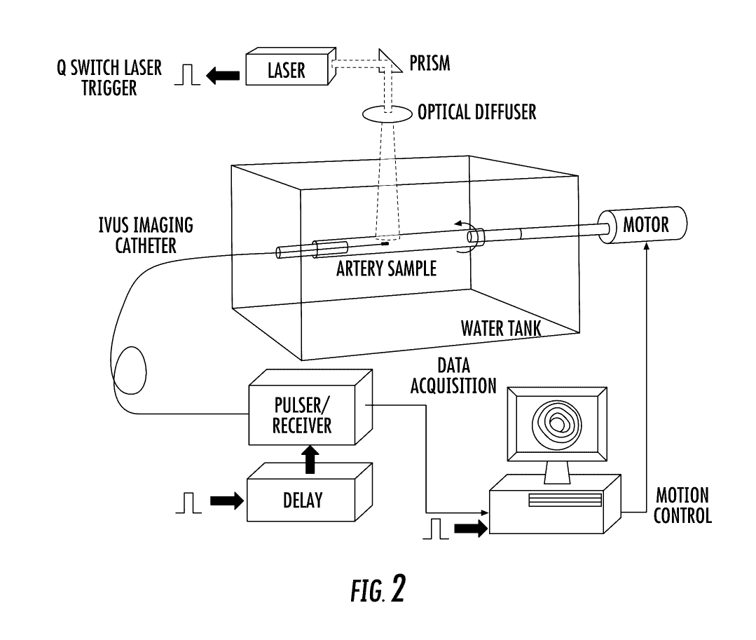

Various components of the combined imaging system were integrated to simultaneously acquire an IVUS and IVPA image. The main components of the IVUS/IVPA imaging system include an optical excitation module needed for photoacoustic imaging, a scanning and imaging module for obtaining co-registered IVUS and IVPA images, an ultrasound signal detection probe and associated electronic components. These components, as used in a laboratory experiment, are illustrated schematically in a FIG. 1a. A block diagram of the laboratory prototype of the combined IVUS/IVPA imaging system is presented in FIG. 1b. The prototype is illustrated more graphically in FIG. 2.

Generally, in photoacoustic imaging, the sample is irradiated with laser pulses of short pulse-width. Generally, pulses 3-10 ns long are used. Pulses of this length (in time) satisfy the acoustic confinement criterion. The selection of an appropriate excitation wavelength is based on the absorption characteristics of the imaging target. In the near-infrared regions, between 2000 and 3000 nm, water is the dominant absorber; the average light penetration depth (the distance through tissue over which diffuse light decreases influence rate to 1/e or 37% of its initial value) varies from about 1 mm to 0.1 mm over this region. At the other end of the spectrum, in the ultraviolet region near 300 nm, the absorption depth is shallow, owing to absorption by cellular macromolecules. In the 400-600 nm range, absorption by blood (hemoglobin) is very strong and residual hemoglobin staining of vessel walls is a strong influence. In the central region between 600-1300 nm, tissue absorption is modest while contrast between tissue components remains high. Therefore, the 500-1100 nm wavelength spectral range is suitable for intravascular photoacoustic imaging since the average optical penetration depth is on the order of several to tens of millimeters.

In our imaging system, an Nd:YAG laser operating at 532 nm or 1064 nm wavelength with a maximum pulse repetition frequency of 20 pulses per second was used. This laser was capable of providing a maximum energy of 24 mJ per pulse. Prior to conducting the imaging experiments, the sample was immersed in a small water tank and fastened to the sample holder at two ends. The sample was irradiated from outside while the IVUS imaging catheter was positioned inside the lumen. The laser beam, originally 2-3 mm in diameter, was broadened using a ground glass optical diffuser such that the laser fluence on the vessel was less than 1 mJ/cm². Hence, the energy was well within the maximum permissible exposure specified by the American National Standards Institute (ANSI). Acoustic and photoacoustic detection.

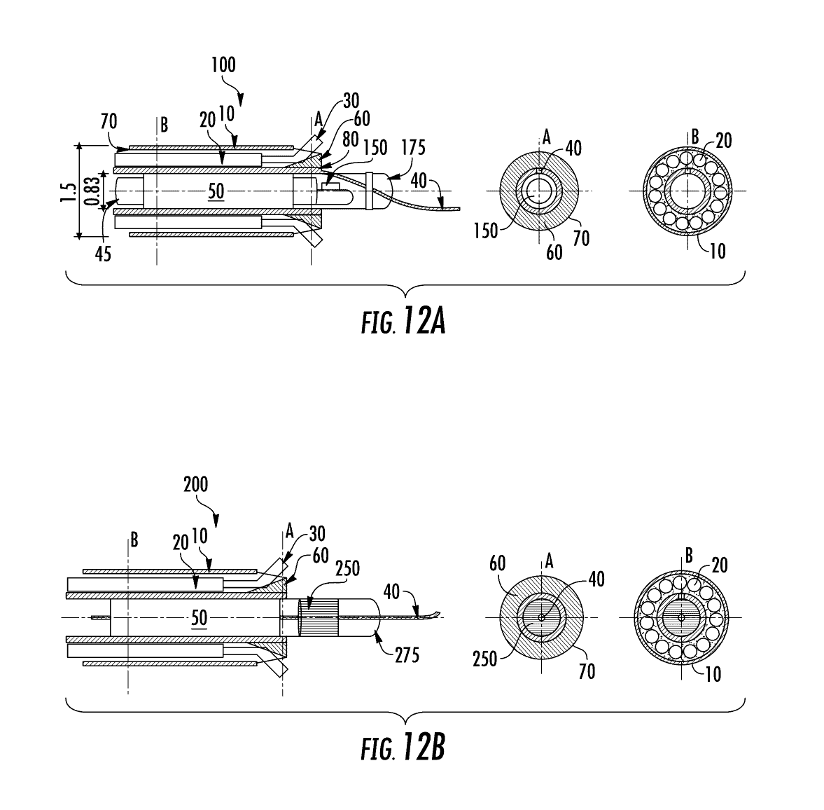

IVUS imaging catheters having acoustic transducer heads with center frequencies of 20 MHz, 30 MHz and 40 MHz were employed as the common probe to detect both the pulse-echo backscattered ultrasound signals (IVUS imaging) and the laser generated photoacoustic waves (IVPA imaging). The sizes of the above catheters were 1.06 mm, 0.96 mm and 0.83 mm in diameter, respectively. The imaging probe 100 (FIG. 12A) contained a single element, unfocused acoustic transducer 150 that required mechanical rotation for scanning the cross-section of the arterial vessel. Indeed, mechanical scanning in IVPA imaging with acquisition following the 20 Hz laser trigger limited the overall scanning time. As seen in FIG. 2, an ultrasonic pulser/receiver was interfaced with the catheter. The pulser electronics were required for transmission of the acoustic pulse for pulse-echo IVUS imaging. The receiver electronics contained an amplifier and a bandpass filter for signal conditioning. The same receiver was used for both IVUS and IVPA imaging modes.

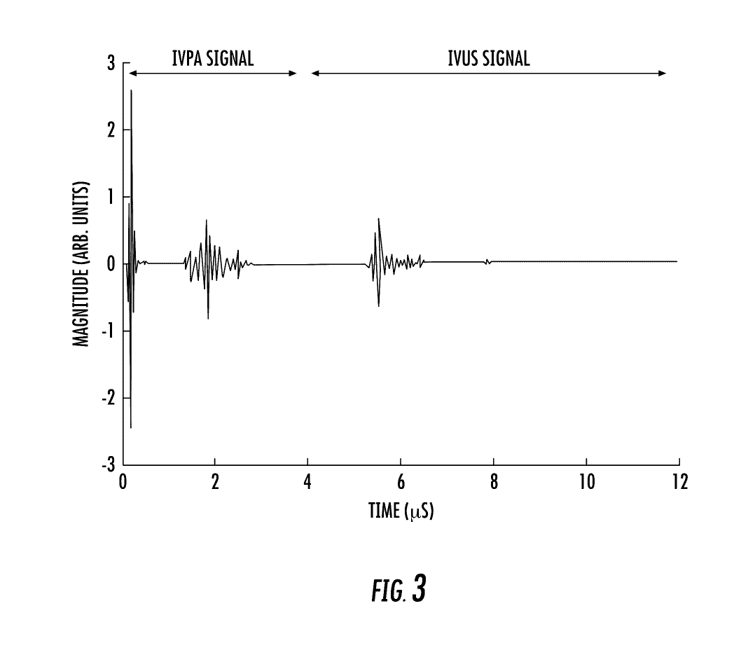

The IVUS imaging catheter 175 was placed inside the vessel sample (either a vessel phantom or arterial tissue); the laser beam irradiated the sample from outside. Since the laser beam in our experimental setup (FIG. 2) was stationary, the transducer and the diffused optical beam were aligned, and the cross-sectional imaging was performed by mechanical rotation of the sample. The overall imaging system was triggered from the laser that was used to initiate IVPA imaging. The same trigger signal, after a delay exceeding the time-of-flight from the deepest structure of the sample, was then sent to the ultrasound pulser. The receiver, therefore, first captured the photoacoustic signal and then the ultrasound pulse-echo signal. An example of these signals (not converted to images) is shown in FIG. 3. Generally, the time-of-flight response of the photoacoustic wave is half that of a pulse-echo IVUS response (“round trip”) due to nearly instantaneous propagation of light.

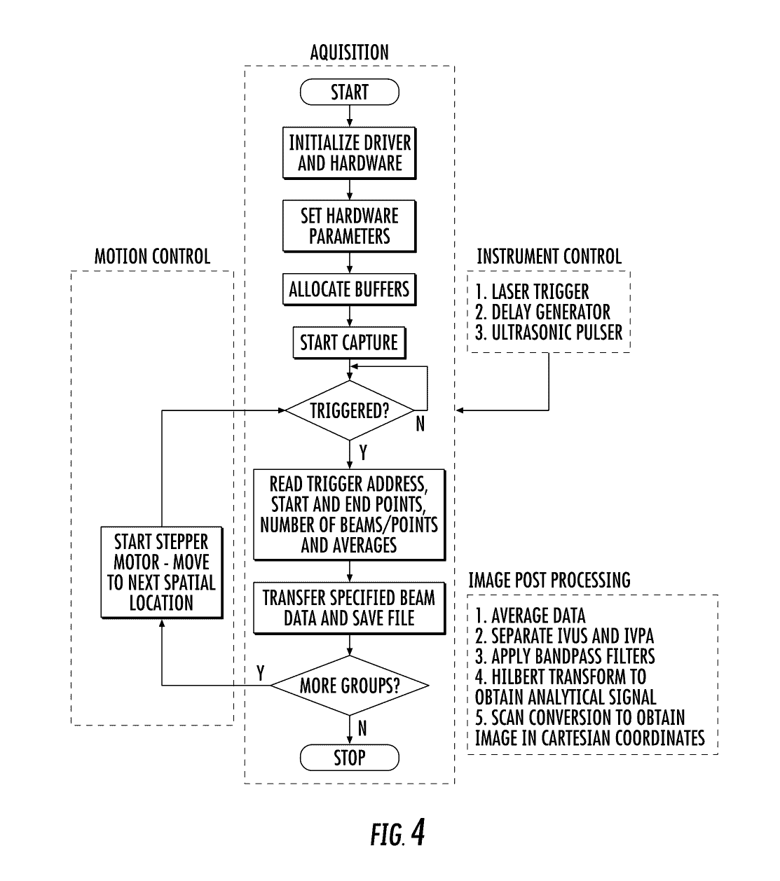

A stepper motor was used to incrementally rotate the cylindrical vessel until IVUS and IVPA signals from the entire cross-section of the sample were obtained. At least 250 A-lines or beams were collected from each cross-section. The term “A-line” refers to a mathematical representation of signals returning from an ultrasound-irradiated target, wherein the magnitude (e.g., amplitude in volts) of the signal is plotted against time. The data were acquired and digitized using a high speed, 14 bit, 200 MHz analog to digital converter. Motion control and rotational scanning, as well as multi-record data acquisition are governed by user-defined algorithms, conveniently embedded in software. Signal averaging and digital filters were applied to improve the signal to noise ratio (SNR). Finally, the signals were scan converted to produce spatially co-registered IVUS and IVPA images. Image acquisition steps and the control system that governs them, together with post-processing steps are summarized in FIG. 4.

In order to test the ability to obtain combined IVUS and IVPA images, imaging experiments were first performed on tissue-mimicking phantoms modeling arterial vessel wall and plaques. The phantoms were prepared using poly vinyl alcohol (PVA). These time-stable phantoms were prepared by mixing 8% polyvinyl alcohol in de-gassed water and heating to 90° C. Varying amounts of additives (silica particles and graphite flakes) were added to the PVA solution to mimic scattering and absorption properties of tissues and associated pathologies. The resulting viscous solution is poured into molds and subjected to alternate periods (12 hrs duration) of freezing and thawing. The results reported here were obtained from a specific cylindrical phantom 100 mm long, 8 mm in diameter, with a 2 mm diameter lumen. Two optically absorbing and scattering inclusions were embedded in the wall of the phantom. Both the vessel wall and the embedded inclusion contained 15 μm silica particles to provide acoustic scattering for IVUS imaging. In addition, to increase optical absorption, the 1.2 mm diameter inclusions had 30 μm fine graphite flakes.

To demonstrate clinical utility of the combined IVUS/IVPA imaging, the experiments were also performed on an ex vivo sample of a rabbit artery. The arterial vessel was excised with the lumen intact and stored in saline for approximately 5 hours before the imaging experiment. The artery was approximately 5 mm in diameter.

In phantom experiments, the IVPA imaging was performed using 1064 nm wavelength, 5 ns pulses. Both IVPA and IVUS imaging utilized imaging catheters operating at 20 MHz, 30 MHz and 40 MHz center frequencies. In tissue experiments, an optical excitation wavelength of 532 nm and a 40 MHz IVUS imaging catheter were used.

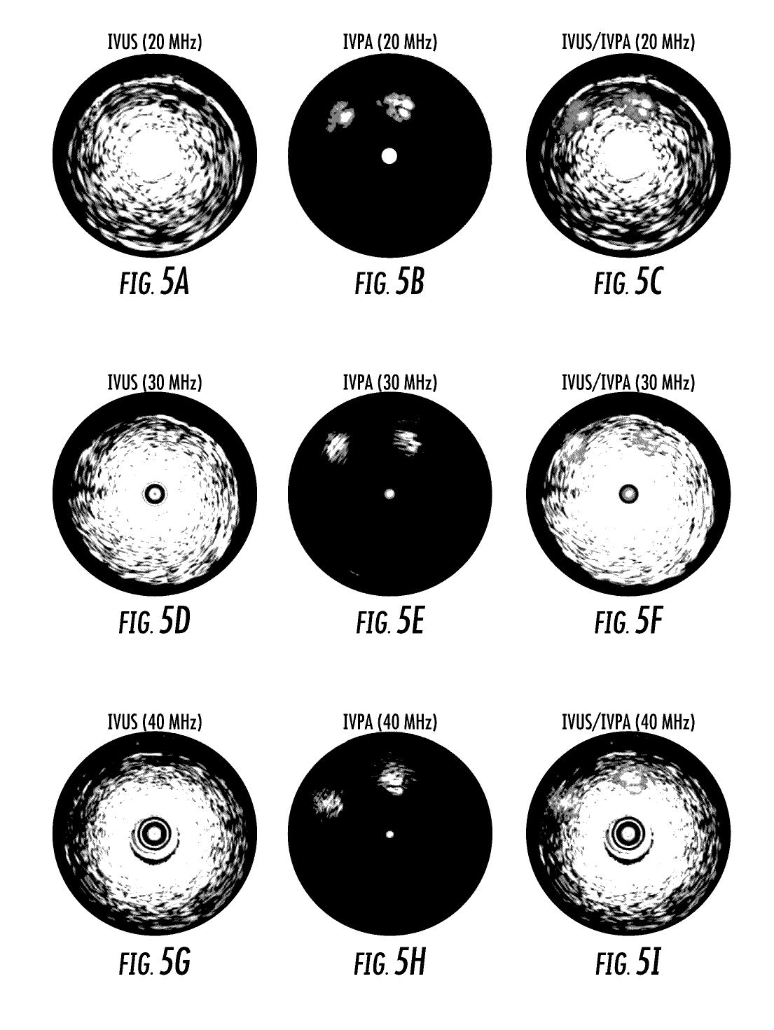

The results of the combined IVUS/IVPA imaging of the vessel phantom with inclusions are presented in FIGS. 5a-5i. All images in FIGS. 5a-5i are displayed over a 9 mm diameter field of view, i.e., each image has a radius of 4.5 mm. These images were obtained from approximately the same cross-section of the phantom. The IVUS images obtained from the 20 MHz, 30 MHz and 40 MHz IVUS imaging catheters are presented in FIGS. 5a, 5d, and 5g, respectively. The bright circle at the center of the image indicates the position of the catheter as evident from the transducer ring-down signal (an artifact in the image driven by a transducer that vibrates for a time in the absence of any incoming signal) and ultrasound echo bouncing off of the plastic sheath covering the transducer. Clearly, the IVUS images show the structure of the phantom, i.e., lumen and the vessel wall. However, IVUS images do not display well the location and extent of the optically absorbing inclusions. As expected, the images obtained with higher frequency probes have better resolution compared to images acquired with IVUS catheters having lower frequency probes. Also visible in all images are artifacts related to uneven rotation of the elastic vessel phantom (e.g., the artifact is located at approximately 7 o'clock in FIG. 5a).

The IVPA images in FIGS. 5b, 5e, and 5h were obtained concurrently with the corresponding IVUS images. The photoacoustic signals from the two inclusions having high optical absorption dominate the image while the other parts of the phantom, which predominantly comprise material that scatters light, have small or no photoacoustic signal. Further, the resolution of the IVPA images is also affected by the frequency of the imaging probe. The 40 MHz probe provides better resolution, as is evident from the IVPA image in FIG. 5h compared to the images presented in FIG. 5b and FIG. 5e (20 MHz and 30 MHz, correspondingly). The circle at the center of the IVPA image results from the direct interaction between light and the surface of the ultrasound transducer.

The synergism of combined IVUS/IVPA imaging is revealed in FIGS. 5c, 5f, and 5i, where photoacoustic signals were overlaid on the IVUS image. The combined images highlight the inclusions in the overall structural context of the phantom, i.e., functional changes in the tissue can be displayed together with anatomical markers of the vessel wall, etc. Further, since the IVUS and IVPA signals are spatially coincident, no image co-registration was required.

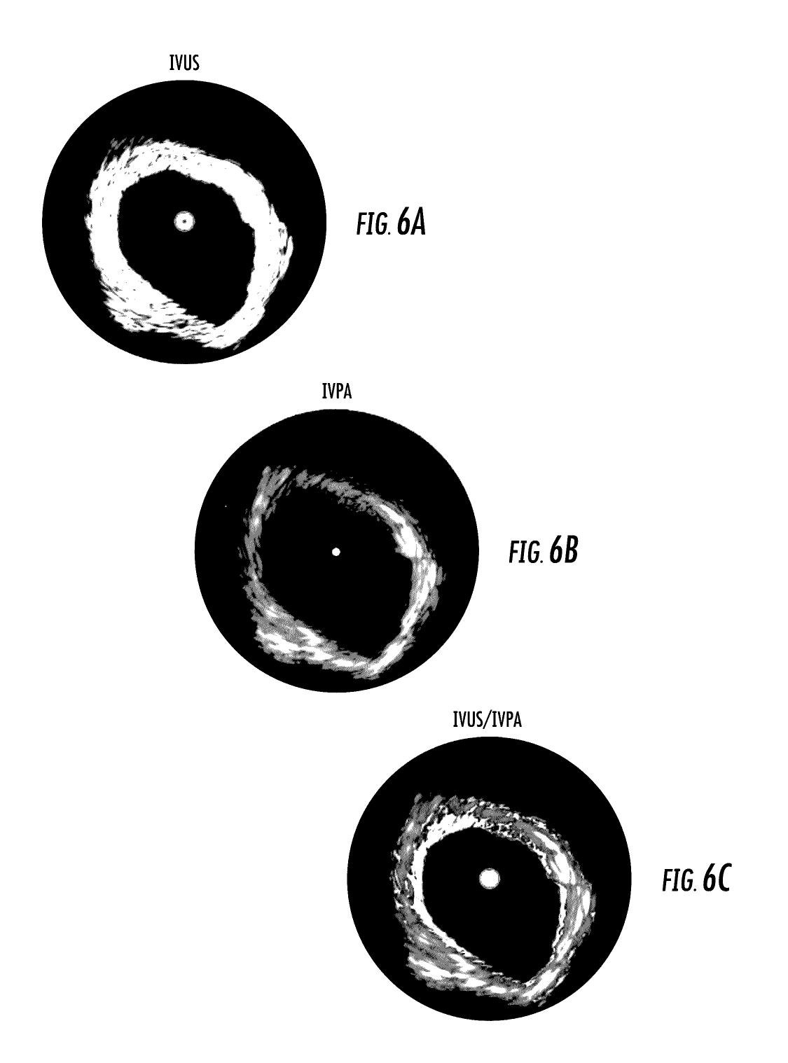

The images presented in FIGS. 6a-c illustrate combined imaging on ex vivo samples of a rabbit artery. The field of view of these images is 6.75 mm in diameter. The photoacoustic signals from the IVPA image in FIG. 6b show excellent correspondence with the IVUS image presented in FIG. 6a. For example, hyperechoic regions at approximately 2 o'clock in the IVPA image correspond well with those in the IVUS image. The combined IVUS/IVPA image of the arterial cross section in FIG. 6c illustrates structural and functional aspects of the combined imaging. Artifacts related to rotation of the tissue sample are evident in these images, e.g., an abrupt change in the images, reminiscent of a knife-cut, located at approximately 3 o'clock.

This Example 1 demonstrates the feasibility of obtaining photoacoustic signals using an IVUS imaging catheter. Further, it shows that the integration of IVPA imaging with IVUS imaging is possible with the combined imaging system. The images presented in FIG. 5 and FIG. 6 emphasize the importance of photoacoustic imaging as a valuable and complementary addition to IVUS imaging.

### Example 2

### Intravascular Photoacoustic Imaging of Atherosclerotic Plaques: Ex Vivo Study Using a Rabbit Model of Atherosclerosis

In Example 1, intravascular photoacoustic (IVPA) imaging was demonstrated using the vessel phantom. Structures having distinct optical absorption characteristics were identified with good contrast in the IVPA images. The results also highlighted the ability of IVPA imaging to provide functional characteristics in addition to anatomical features exhibited by the intravascular ultrasound (IVUS) imaging. The initial IVPA images of the excised aorta samples show that photoacoustic signals can be obtained from highly scattering vessel wall structures. In this Example 2, we further investigated the ability of IVPA imaging to differentiate plaques through ex vivo studies on the aorta obtained from a rabbit model of atherosclerosis. In addition, we performed experiments to investigate the challenges associated with the in vivo implementation of IVPA imaging. Specifically, we analyzed the impact of optical absorption of blood on the ability of photoacoustic imaging to detect plaques, and considered the configuration of the imaging catheter needed form clinical implementation of IVUS assisted IVPA imaging.

Rabbits fed on a high cholesterol diet are appropriate models for the study of atherosclerosis (Overturf, M. et al., “In vivo model system: the choice of experimental model for analysis of lipoproteins and atherosclerosis,” Curr. Opin. Lipidology 2: 179-185, 1992). In rabbits susceptible to hypercholesterolemia, lesion development starts with the early increase of focal arterial low density lipoproteins, followed by sub-endothelial deposits of extracellular lipids and cytosolic lipid droplets of smooth muscle cells. The initial fatty streaks quickly develop into intimal lesions containing macrophage derived lipid-filled foam cells. In three months, the lesion progresses to advanced fatty streaks with equal number of foam cells and spindle shaped cells and finally to more complex fibrous plaques and advanced atheromatous lesions (Guyton, J. R. et al., “Early extracellular lipid deposits in aorta of cholesterol-fed rabbits,” Am. J. Pathol. 141: 925-936, 1992).

The degree and types of lesions are dependent on the dietary regimen administered to the rabbit models. A high cholesterol diet (1-4% or more) result in rapid development of lesions with a lipid core and macrophage enriched foamy lesions. The lesions originate in the aortic arch and are also found in the thoracic aorta. A milder dietary regimen (<0.2% cholesterol) fed over a longer period of time (5-6 months) induce more complex lesions that more closely resemble those found in humans. The lesions have extracellular matrix development, large number of smooth muscle cells, and cholesterol crystals typical of advanced human atherosclerotic and vulnerable plaques (Daley, S. J. et al., “Cholesterol-fed and casein-fed rabbit models of atherosclerosis, Parts 1 and 2: Differing lesion area of volume despite equal plasma cholesterol levels,” Arterioscler. Thromb. 14: 95-114, 1994; Rosenfeld, M. E. et al. “Lipid composition of aorta of Watanabe heritable hyperlipemic and comparably hypercholesterolemic rabbits,” Arteriosclerosis 8: 338-347, 1988). These lesions may end up as mixed plaque with fibrous and cellular components in addition to lipid deposits. In our imaging study, one year old New Zealand rabbits subjected to a mild cholesterol diet of 0.15% cholesterol spread over a longer period of time (12 months) were employed. In addition, a rabbit kept for the same time period under normal diet conditions was used as the control sample in imaging experiments.

The rabbits were pre-anesthetized and intubated with a 3.5 French endotracheal tube and placed on a small animal ventilator of 95% oxygen. During the surgical procedure, marcaine was administered topically. Through a cut in the right femoral artery a 4 French NIH catheter was used for performing an aortic angiogram. Then, a 0.014″ guide wire 40 was inserted to direct the Boston Scientific IVUS imaging catheter (iSight™) up to the aortic arch. The location of the IVUS imaging transducer was determined from the contrast injected angiogram. Following the positioning of the IVUS catheter, a “pull back” IVUS imaging was performed to identify plaque deposition along the aorta from the thoracic to the renal end of the aorta. The pullback data were recorded and the location of the lesions was noted in the context of anatomical landmarks and major arterial branches. The rabbit was sacrificed using super saturated potassium chloride and the aorta was excised in full length. The branches were marked with sutures and the excised aorta was stored in saline for about 5 hours. Several segments with potential plaques were then made available for the ex vivo imaging using the integrated IVUS/IVPA imaging system described in Example 1.

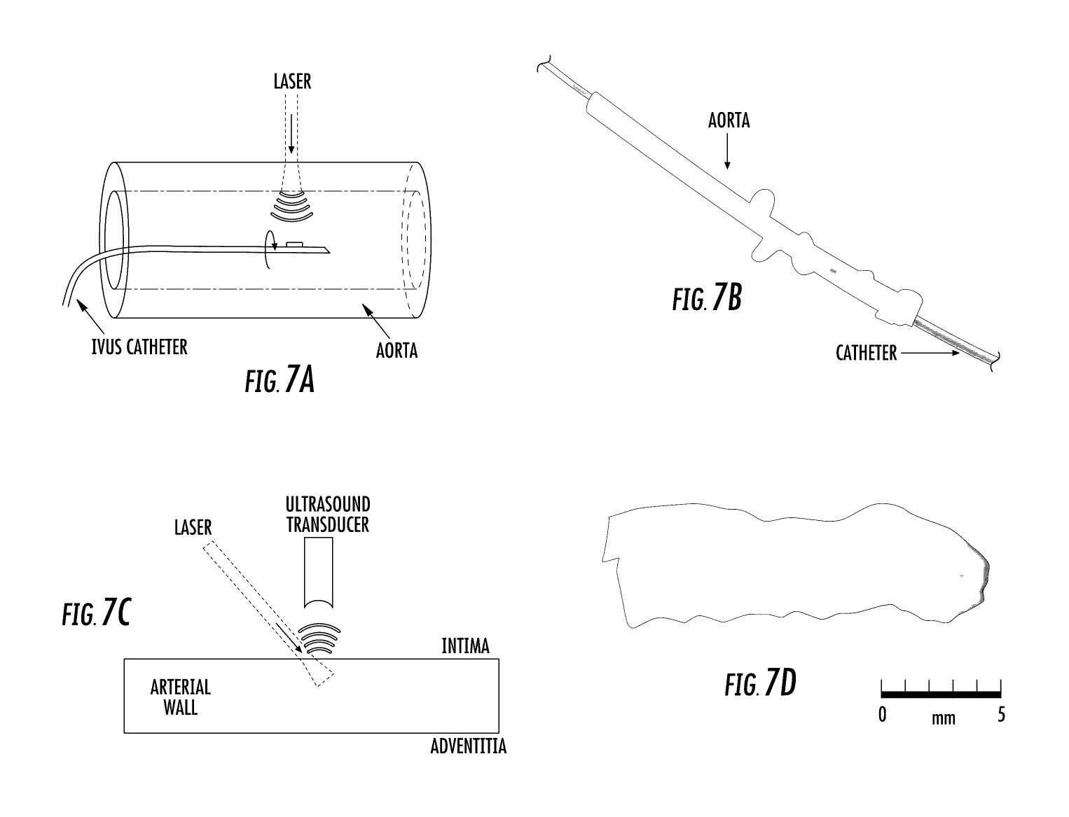

Briefly, the excised aorta was washed in saline to remove any blood clots in the lumen, cut into 6 cm long segments and secured in a custom-built water tank. To simplify the imaging procedure, the photoacoustic imaging was performed in a forward mode configuration where the optical excitation and photoacoustic detection are on either side of the wall of the aorta (FIG. 7A). The photograph of a segment of the aorta with the IVUS imaging catheter placed in the lumen is shown in FIG. 7B. The Q-switched Nd:YAG laser provided laser pulses at a repetition rate of 20 Hz and a maximum energy of 24 mJ per pulse at 532 nm. The energy fluence was minimized to approximately 1 mJ/cm² by broadening the beam diameter using a ground glass diffuser. The photoacoustic transients were detected using a single element 40 MHz, 2.5 French, IVUS imaging catheter 175. Simultaneous IVUS and IVPA signals were obtained using the integrated imaging system (Sethuraman, S. et al., “Development of a combined intravascular ultrasound and photoacoustic imaging system,” Proceedings of the 2006 SPIE Photonics West Symposium: Photons Plus Ultrasound: Imaging and Sensing 6086: Fl-Fl 0, 2006; Sethuraman, S. et al., op. cit.). A motion control system was used to incrementally rotate the sample and 250 A-lines were acquired for one complete rotation of the sample. Depth dependent compensation of the photoacoustic response was applied to account for the attenuation of light through the tissue. Finally, the signals were bandpass filtered to remove noise and scan converted to display images in the Cartesian system of coordinates.

As opposed to the ex vivo IVPA imaging performed in the forward mode (FIG. 7A), experiments were also performed in the backward imaging mode where the imaging transducer and the optical illumination were on the same side of the tissue (FIG. 7C). The ultrasound echo and photoacoustic ultrasound experiments were conducted using a probe 100 (FIG. 12A) with a single element, focused, 4 mm aperture, 5.8 mm focal length, 48 MHz ultrasound transducer 150. The optical illumination was provided by a pulsed laser operating at 532 nm wavelength and delivered to the tissue from the top using prisms 60. A carotid artery, obtained from the atherosclerotic rabbit used for the intravascular imaging experiments, was utilized in these studies. The excised artery was cut along the longitudinal axis of the vessel, opened and placed flat in the water tank such that the intimal side of the vessel along with the plaques faced the probe 100. The acoustic detector was placed above the excised carotid artery at a distance of approximately 5 mm so that the arterial tissue layers lie within the focus of the transducer. Following approximate alignment of the laser spot with the ultrasound detector, IVUS and IVPA scanning were simultaneously performed on the tissue sample by incrementally moving the probe 100. Ultrasound echo and photoacoustic images were obtained from the artery shown in FIG. 7D with a scan length measuring 15 mm longitudinally along the vessel. The radiofrequency signals were acquired at a sampling rate of 500 MHz, and processed off-line to generate spatially co-registered photoacoustic and ultrasound echo images of the vessel wall tissue.

The elevated attenuation of both laser energy and photoacoustic transients is expected to occur in the presence of blood between the photoacoustic catheter probe and the wall of the arteries. The ultrasound attenuation in blood is manageable at the IVUS frequencies, but the elevated absorption of photons in blood could produce two undesired effects. First, the photoacoustic signals from the tissue are likely to be weaker and may not have desired signal-to-noise ratio thus degrading the quality of the photoacoustic image. Second, strong photoacoustic response from the blood-stained arterial wall could overlap and corrupt the photoacoustic signals from the arterial wall and plaque. Therefore, to investigate the influence of the luminal blood in the photoacoustic imaging, we compared the photoacoustic response from the excised carotid artery immersed in a saline bath and in slightly diluted blood. The blood contained heparin as an anti-coagulant administered prior to sacrificing the rabbit. To increase light penetration in blood, the photoacoustic imaging probe 100 was used with a tunable pulsed laser source operating at 700 nm wavelength. The ultrasound echo and photoacoustic imaging was performed by mechanically scanning the imaging probe over an area containing visually identifiable plaques. The photoacoustic signals from the blood were identified and eliminated using the ultrasound echo image. Indeed, IVUS reveals the structural content in the image where solid tissue can be easily recognized. Further, a user selected gain was applied to the photoacoustic signals to compensate for depth dependent variation of the laser fluence.

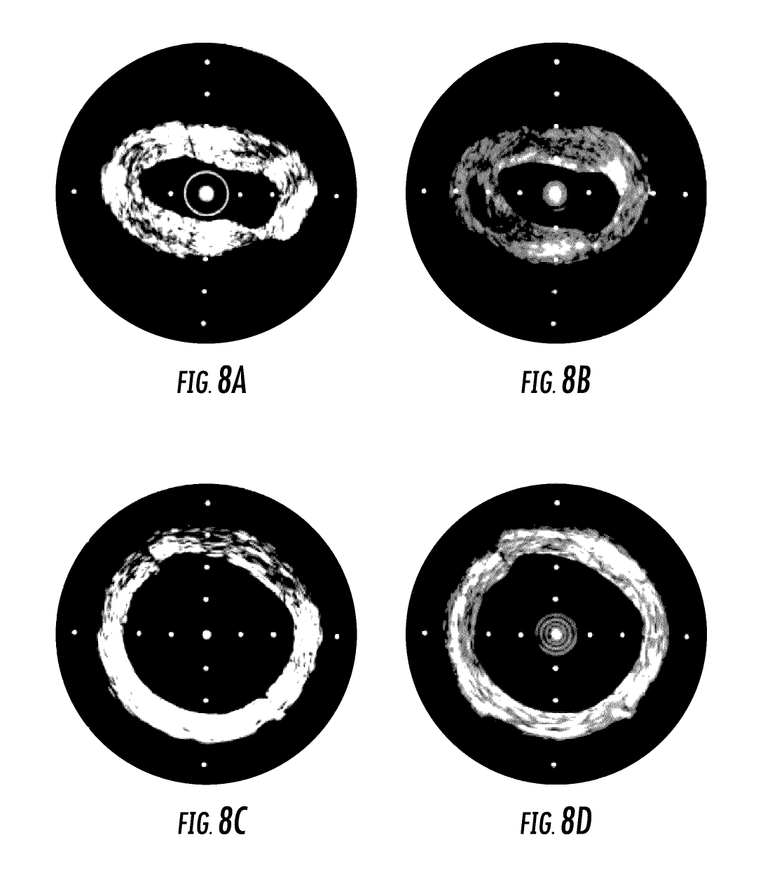

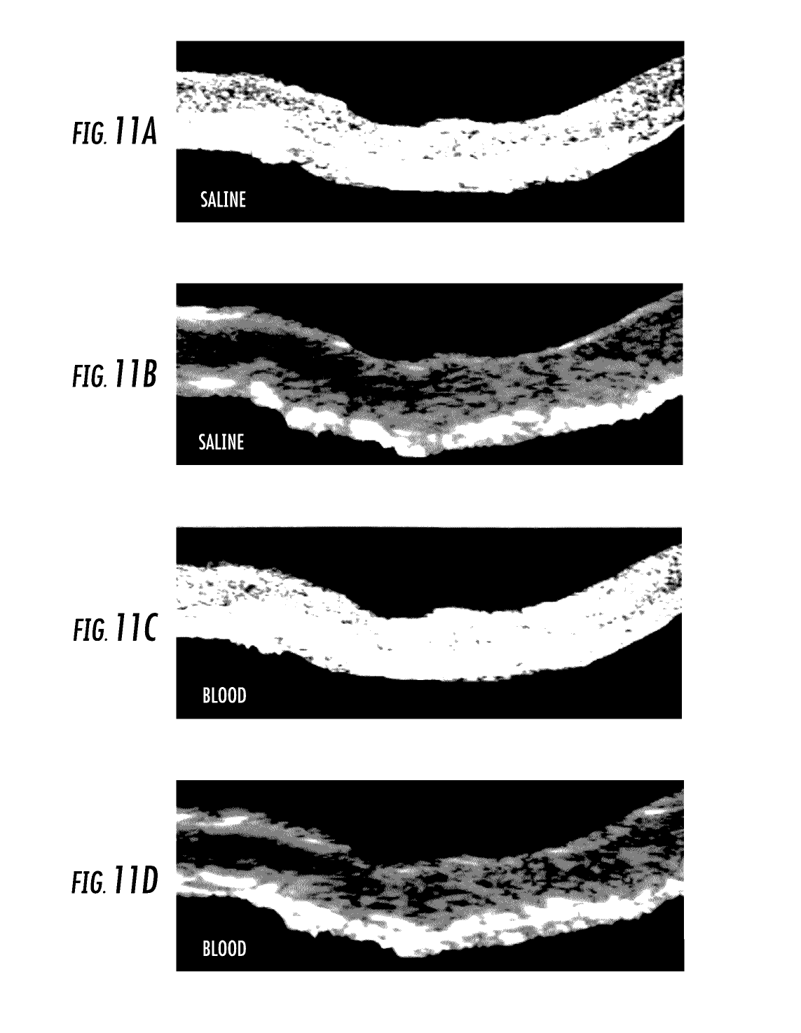

The results of the ex vivo IVUS/IVPA imaging of the plaque laden and normal rabbit aortas are presented in FIGS. 8A-D. The IVUS image in FIG. 8A clearly shows the decrease in the diameter of the lumen. Further, a change in the ultrasound speckle characteristics gives an indication of the plaque deposition all along the intima of the vessel. However, the extent and composition of the plaque is not well understood from the IVUS image. On the other hand, the IVPA image in FIG. 8B obtained from the same location on the vessel as the IVUS image shows some distinct characteristics. First, the most striking feature in the IVPA image is the presence of hypoechoic regions between 7 o'clock and 9 o'clock and also between 10 o'clock and 12 o'clock. Second, there is a measurable photoacoustic response from the superficial region located between 9 o'clock and 1 o'clock. This lipid-rich region of the plaque could contain fibrous cap and infiltrated macrophage cells. The other regions of the vessel exhibit uniform or hyperechoic photoacoustic signals indicating normal aortic tissue. The IVUS and IVPA images, presented in FIG. 8(C-D), indicate a larger (5 mm diameter) lumen of the normal aorta with a thin (0.8 mm) vessel wall. The IVPA image further details homogeneous photoacoustic response from the fibrous components of the normal aorta. Also noted in the images are artifacts (e.g., at 11:30 o'clock in FIG. 8A and 11 o'clock in FIG. 8C) caused by irregular rotation of the soft arterial tissue.

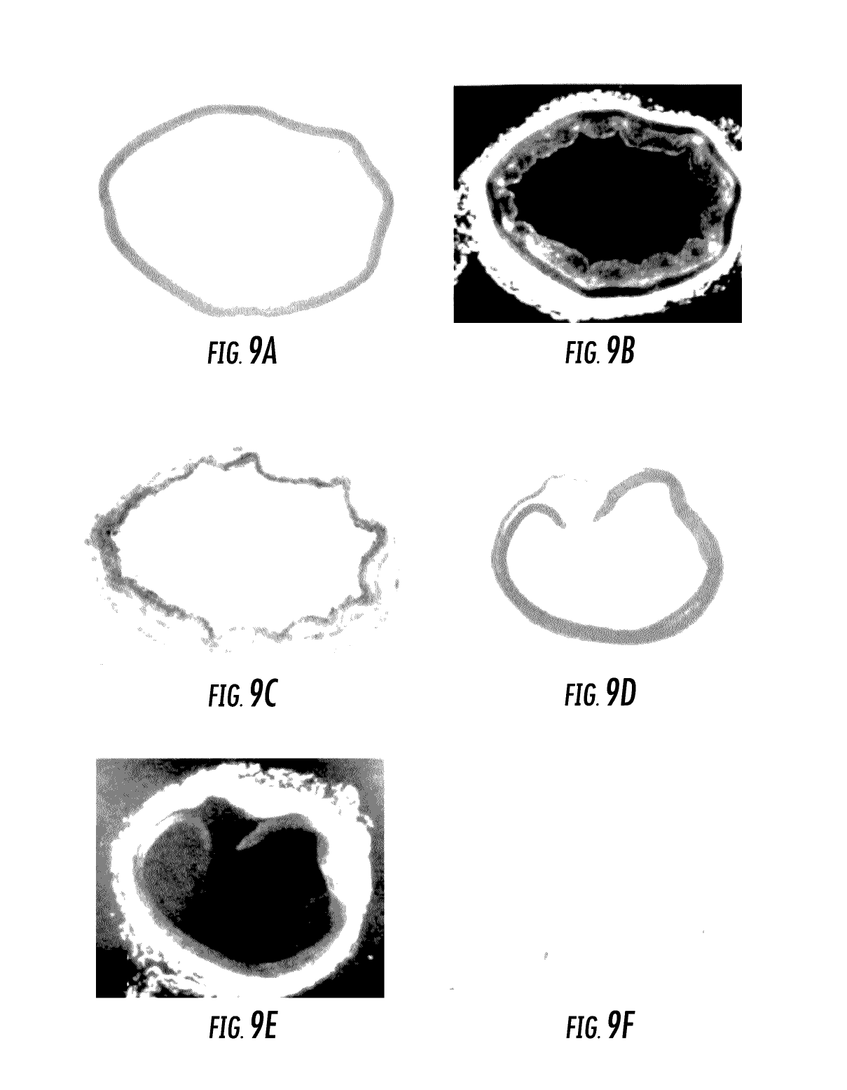

To confirm the results obtained from the IVUS/IVPA imaging, histological analysis was performed at the imaged cross-section. The histology images of the atherosclerotic and normal aorta are presented in FIGS. 9A-F. The H&E stained image in FIG. 9A indicates a thick intima resulting from the plaque accumulation all along the vessel. The presence of focal accumulation of thick collagen is indicated by spots in FIG. 9B in the Picrosirius red stained image obtained under a polarization microscope. This image also shows the presence of the thin collagen in the region near the intima-media boundary. In addition, macrophage cells in response to increase of low density lipoproteins are seen in the RAM-11 stained image in FIG. 9C. In contrast, the H&E stained image in FIG. 9D is characterized by a thin intima composed of an endothelial layer with an underlying media composed of elastic fibers and smooth muscle cells. The lack of intimal thickening preserved the luminal size. Further, the Picrosirius red stained image in FIG. 9E illustrates the presence of thin collagen and RAM-11 stained image in FIG. 9F did not stain positively for macrophages.

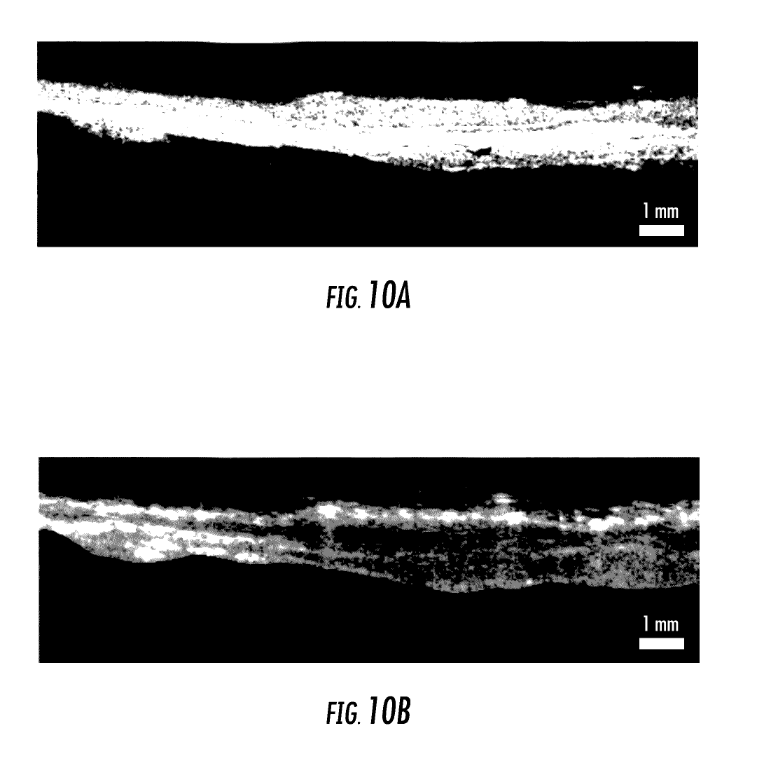

The photoacoustic images in the backward mode imaging configuration and the corresponding ultrasound echo image is presented in FIG. 10A and FIG. 10B. The B-Scan (that is, the displayed image) of the carotid artery, presented in FIG. 10A, clearly outlines the thickened intima (indicator of plaque), media, adventitia and the underlying fat. The image in FIG. 10B shows the photoacoustic response from the same carotid artery. The plaque in this image can be identified as dark regions in the extended intima. Further, the fibrous tissue above the plaque show increased photoacoustic response indicating higher absorption. The distance between the transducer and the tissue in the backward mode was chosen such that the tissue lies within the focal region of the transducer. In the clinical setting, the distance between the imaging catheter and the arterial wall is expected to be similar to the distance used in our studies. Clearly, the IVPA image and photoacoustic image obtained using forward and backward imaging modes, respectively, are similar. Indeed, vessel wall and plaque have the same features on both images. Therefore, the change in imaging configuration did not have significant effect on the photoacoustic images and the plaque was detected in both the forward and backward imaging configurations.

Furthermore, the plaque could also be reliably identified in the presence of blood. The 6.4 mm by 2.1 mm images presented in FIGS. 11A-D illustrate the ultrasound echo and photoacoustic images obtained from tissue sample immersed in saline and blood. The B-Scan images of the cross-section of the carotid artery (in saline and blood) are presented in FIGS. 11A and 11(C). The images are, as expected, very similar and clearly show a uniform thickening of the intima all along the cross-section. However, there is a definite deterioration of the ultrasound speckles in the extreme left and right regions of the images most likely caused by the presence of lipids. This observation is supplemented by the presence of hypoechoic regions in the same areas in the photoacoustic images in FIGS. 11B and 11D. The magnitude of the photoacoustic response from the tissue in the presence of blood shown in FIG. 11D was lesser than the response in the presence of saline. Indeed, the attenuation of light through blood leads to a decrease in the laser energy incident on the artery. However, the depth dependent correction of the photoacoustic response in the artery to compensate for the light attenuation by blood resulted in an image similar to that obtained in saline.

The ex vivo photoacoustic imaging results indicate that the plaques in the artery can be detected and possibly differentiated. The lipid in lipid-filled plaques in all cases manifested itself as dark regions due to lesser optical absorption at 532 nm. Indeed, the optical absorption coefficient of fat at 532 nm is low and has been shown to be approximately 0.01 cm⁻¹ (van Veen, R.L.P. a. S., et al., “Determination of visible near-IR absorption coefficients of mammalian fat using time- and spatially resolved diffuse reflectance and transmission spectroscopy,” J Biomed. Optics 10: 540041-540046, 2005). Also common in these images is the presence of strong photoacoustic signals from the superficial layer above the lipid. The spatial correspondence of the expression of RAM-11 (an antigen associated with macrophages) and the strength of the photoacoustic signal could indicate the presence of light absorbing macrophages. The location of these hyperechoic signals also correlates well with the fibrous cap containing collagen fibers indicated by the Picrosirius red stained histology images. Further, since the histology indicates the plaque to be fibro-cellular, the magnitude of the photoacoustic signal could be affected by the collagen as well as infiltrating macrophages and smooth muscle cells.

The ability to obtain photoacoustic response and detect plaque using a 700 nm laser illumination in the presence of blood (FIG. 11D) suggests that clinical implementation of intravascular photoacoustic imaging is possible. Indeed, the absorption by blood is relatively low in the optical diagnostic window of 700 nm-900 nm. Therefore, selecting the appropriate wavelength is critical for IVPA imaging. Apart from minimizing blood absorption, photoacoustic imaging at a wavelength of 900 nm may increase lipid absorption (Tromberg, B J. et al., “Non-invasive in vivo characterization of breast tumors using photon migration spectroscopy,” Neoplasia 2: 26-40, 2000). The imaging results from this study suggest that a multi-wavelength interrogation of the tissue in the optical diagnostic window is likely to increase the contrast between the various constituents of plaques, improve plaque detection and provide sufficient penetration of light through blood and tissue.

The ex vivo tissue study supplemented with the histopathological analysis confirmed that IVPA imaging can detect plaques. The photoacoustic images obtained from the aorta and carotid artery from an atherosclerotic rabbit is consistent in identifying the presence of foamy macrophage lesions. The photoacoustic images provided information supplementary to that obtained from the ultrasound echo images. Therefore, the combination of IVPA imaging with IVUS imaging is useful and is expected to improve the clinical utility of IVUS imaging. Further, the results of the photoacoustic imaging obtained in clinically relevant environment suggest that in vivo implementation of IVPA imaging is possible.

### Example 3

### Combined IVUS/IVPA Imaging In Vivo

In this Example 3, an integrated IVUS/IVPA imaging catheter 100 suitable for clinical use is made by surrounding an IVUS catheter (iSight™ in the single-element device 175 in this example, the Avanar® FIX in the multielement device 275) with an array of optical fibers 20, which array is itself surrounded by an outer sheath 10 fabricated with a flexible plastic material to create a combination catheter 100 (FIG. 12A). A copolymer of polyoxymethylene and polyurethane is exemplary (see U.S. Patent Publication 2003/0167051, incorporated herein in its entirety by reference for all purposes). The arrayed optical fiber bundles 20 are embedded or “potted” in a glue 70. The glue is capable of adhering to the material of the inner sheath 80, the outer sheath 10 and the outer surfaces of the fiberoptic bundles 20 and, after curing, has about the same degree of flexibility as these materials. Each fiber bundle 20 originates proximally at an interface with a laser light source and ends distally in an annular cavity defined by the distal ends of the fiber bundles 20 and by the inner aspect of the wall of the outer sheath 10 and the outer aspect of the wall of the sheath that surrounds the electrical leads 50 of the IVUS catheter assembly (“inner sheath”). The inner sheath 80 extends distally beyond the distal terminus of the outer sheath 10. Affixed to the outer aspect of this distal region of the inner sheath 80 is affixed an annular array of prisms 60. Each fiberoptic bundle 20 is configured and disposed within the integrated IVUS/IVPA catheter 100 to be capable of emitting a beam of light through the annular cavity onto the surface of an affixed prism 60, which prism is configured and disposed to deflect the light beam 30 radially outward from the long axis of the integrated catheter 100 to illuminate the walls of the vessel in which the catheter dwells. The integrated catheter 100 is interfaced with the IVUS/IVPA console containing a pulsed laser device and electronic integrated circuits incorporating the functionalities that control ultrasonic pulsing, ultrasonic and photoacoustic signal conditioning, and user-defined delay mechanisms. The entire system is controlled through a console containing user controllable features that include, IVUS-IVPA-spectroscopic IVPA imaging modes, change of laser energy and wavelengths, attenuation and time gain compensation of signals.

The integrated imaging probe 100 consisting of an IVUS catheter 175 equipped with an ultrasound transducer 150, along with an optical fiber light delivery assembly, is placed in the lumen of the artery. In such an “inside-out” configuration, the combined imaging system is intravascular for both ultrasound echo and photoacoustic imaging. In this configuration, the IVUS imaging probe 150 is rotated as it sends and receives signals. Alternatively, no mechanical rotation is necessary if an array-based IVUS system 275 is employed. A clinically viable imaging system wherein a fiber optic light delivery system is integrated with an IVUS imaging catheter 275 to permit combined IVUS/IVPA imaging within the lumen of the vessel. The integrated system 200 is exemplified in FIG. 12B.

Several light delivery probes are discussed in the literature and are currently investigated for a wide range of optical imaging and therapeutic techniques (P. C. Beard, F. Perennes, E. Draguioti, and T. N. Mills, “Optical fiber photoacoustic-photothermal probe,” Optics Letters, vol. 23, pp. 1235-1237, 1998).

To minimize undesired attenuation of laser energy by optical absorption in luminal blood before the energy reaches the vessel wall, one may flush the vessel lumen with saline or other clearing agents. A more clinically desirable approach is to identify the optimal excitation wavelength for IVPA imaging by performing spectroscopic photoacoustic imaging (P. C. Beard and T. N. Mills, “Characterization of post mortem arterial tissue using time-resolved photoacoustic spectroscopy at 436, 461 and 532 nm,” Phys Med Biol, vol. 42, pp. 177-98, 1997; A. A. Oraevsky, V. S. Letokhov, S. E. Ragimov, V. G. Omel Yanenko, A. A. Belyaev, B. V. Shekhonin, and R. S. Akchurin, “Spectral properties of human atherosclerotic blood vessel walls,” Laser Life Sci., vol. 2, pp. 275-88, 1988). The technique also differentiates certain specific structures in plaque and, by providing a higher signal to noise ratio, leads to a better assessment of plaque composition.

Another configuration for intravascular IVUS imaging catheters is a “forward looking” transducer. These catheters are helpful in generating 2D planes and 3D volumes in heavily occluded vessels and extremely important in guiding interventions. An annular array placed at the catheter tip has been developed that minimizes the interference from the guide wire (Y. Wang, D. N. Stephens, and M. O'Donnell, “Optimizing the beam pattern of a forward-viewing ring-annular ultrasound array for intravascular imaging,” IEEE Trans Ultrason Ferroelectr Freq Control, vol. 49, pp. 1652-64, 2002). Capacitive micro-machined ultrasound transducer (cMUT) technology is being widely explored for use in forward-looking catheter configuration (J. G. Knight and F. L. Degertekin, “Fabrication and characterization of cMUTs for forward looking intravascular ultrasound imaging,” Proc. IEEE Ultrason. Symp., pp. 577-580, 2002).

Numerous arrays can be fabricated on a single silicon wafer that would be broadband with higher sensitivity compared to a piezo electric transducer (U. Demirci, A. S. Ergun, O. Oralkan, M. Karaman, and B. T. Khuri-Yakub, “Forward-viewing CMUT arrays for medical imaging,” IEEE Trans Ultrason Ferroelectr Freq Control, vol. 51, pp. 887-95, 2004).

Combined IVUS and IVPA imaging system can also incorporate ultrasound based intravascular elasticity imaging or intravascular palpography (C. L. de Korte, G. Pasterkamp, A. F. van der Steen, H. A. Woutman, and N. Born, “Characterization of plaque components with intravascular ultrasound elastography in human femoral and coronary arteries in vitro,” Circulation, vol. 102, pp. 617-23, 2000). Indeed, the acquisition of a large number of IVUS beams would help in obtaining simultaneous strain images for differentiating tissue structures based on mechanical contrast. Hence, it is possible to envision a multi-technique ultrasound based intravascular imaging system that would help in the detection and differentiation of atherosclerosis (S. Sethuraman, S. R. Aglyamov, J. H. Amirian, R. W. Smalling, and S. Y. Emelianov, “An integrated ultrasound-based intravascular imaging of atherosclerosis,” Proc. of the fourth international conference on the ultrasonic measurement and imaging of tissue elasticity, pp. 69, 2005).

In the in vivo implementation of IVPA imaging in this Example 3, the integrated IVUS/IVPA probe 100 (FIG. 12A) is inserted into the aorta via the femoral artery through a femoral cut. The catheter 175 is positioned in the aorta close to the aortic arch with the help of a contrast injected angiogram. Following the positioning of the IVUS catheter 175, multiple longitudinal pull-back imaging is performed to interrogate the artery ultrasonically. The real-time IVUS images are obtained and the position of the areas of suspected plaque deposition are mapped. Following IVUS examination of the artery, the catheter 100 is positioned at the areas noted as being suspect and the IVPA imaging mode is incorporated. At the user's discretion, a given segment of the artery may be IVUS-imaged and IVPA imaged before the catheter 175 is pulled or pushed to the next segment. The photoacoustic response is acquired and displayed super-imposed on the IVUS cross section. Specifically, the IVPA imaging is obtained within an optical excitation range of 680 nm-1000 nm. Where the IVPA response is not significant, laser beam energy and wavelength is modified to obtain images having a useful signal to noise ratio. The system also contains ultrasound-based temperature monitoring algorithms to approximately estimate the temperature increase in the artery at a specific laser energy. Indeed, the temperature estimation is useful to limit the level of optical energy and ensure safety.

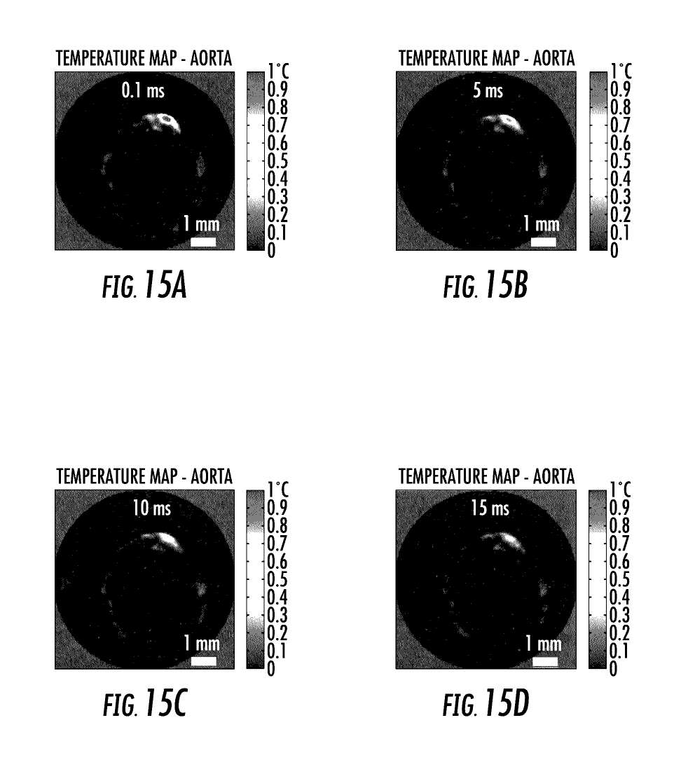

To demonstrate the safety of the method, we utilized an ultrasound based technique to measure to measure the temperature increase in the aorta resulting from laser excitation. The change in the speed of sound due to temperature increase would change the time of flight response in the IVUS signals. Therefore, an analysis of the apparent change in the nature of the IVUS echoes would help us to obtain the temperature. This technique of combined IVUS/IVPA imaging helped us to address the thermal safety of IVPA imaging. The maximum temperature increase observed (more laser energy was utilized than necessary) was 1.1° C. The results of the technique are shown in image form in FIGS. 15A-15D.

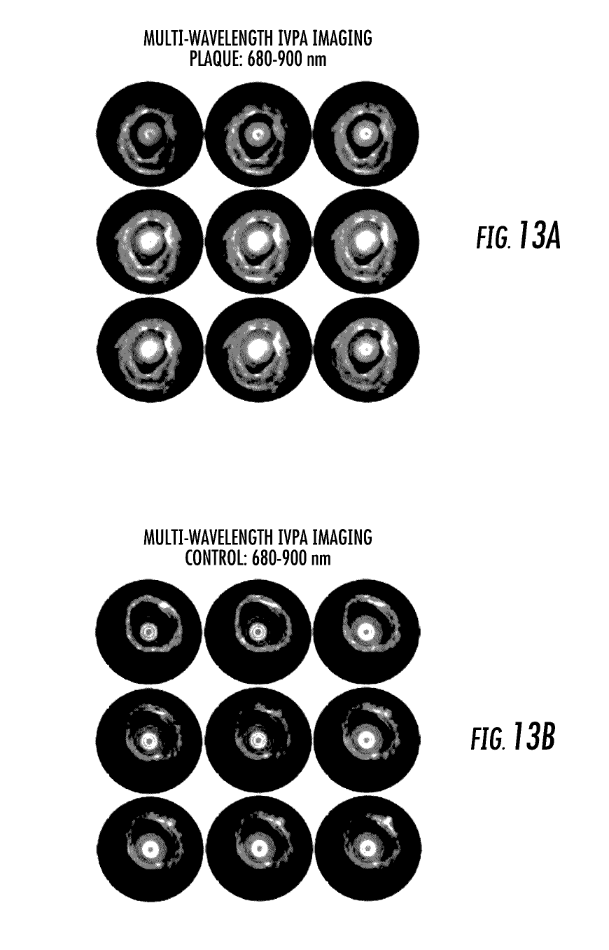

The IVPA image is said to be “spectroscopic” because the IVPA imaging is performed at multiple wavelengths, specifically, in this example, 680 nm-900 nm at increments of 20 nm (FIGS. 13A and 13B). This further enriches the image by adding gradations to it. While applicants will not be bound by any theory explaining the mechanism underlying this effect, it is thought that because the amplitude of the photoacoustic response is a function of the optical absorption coefficient of the imaged object, variations in optical absorption coefficients within the object (that is, variations in the image of the object) is a function of the wavelength of the laser illumination. Thus, spectroscopic illumination “brings out” different pixel values depending upon the composition of the imaged tissue.

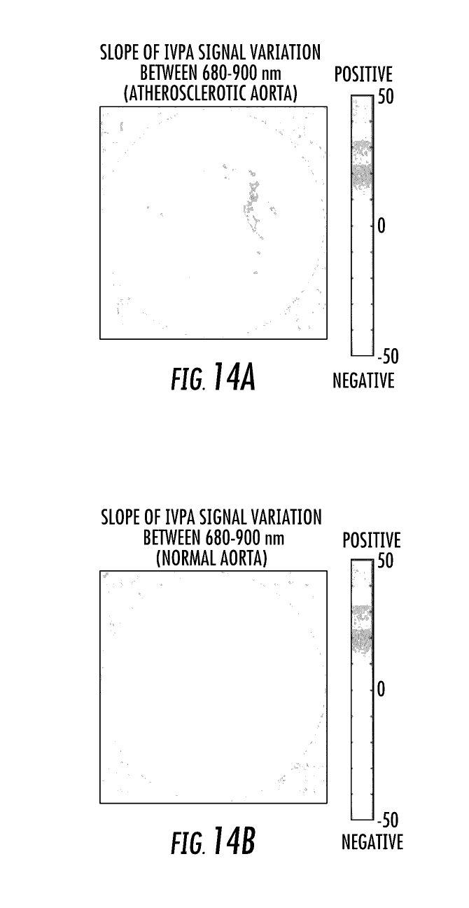

In the above-mentioned spectroscopic mode, a polynomial fit is performed (implemented in the system) to obtain the functional variation of photoacoustic signal with wavelength. A first derivative of the spectral function is indicative of the specific plaque composition as seen in the derivative image. For example, in FIG. 14A the plaque containing extensive lipid deposition is indicated by areas have positive derivative values (FIG. 14A). The increase in optical absorption by lipids from 680 nm to 900 nm contributed to the increase in photoacoustic signal. The normal tissue in the image is indicated by negligible variation in photoacoustic signal in the wavelength range 680 nm-900 nm (FIG. 14B). Hence, the different pixel values display the first derivative values and highlight the heterogeneous nature of the plaque.

1-19. (canceled)

20. A method for creating intravascular ultrasound (IVUS) and intravascular photoacoustic (IVPA) images of a blood vessel, the method comprising:
providing an IVUS/IVPA probe, wherein the IVUS/IVPA probe comprises:
a rotatable ultrasound transducer configured to transmit and receive ultrasound in a general direction,
an optical excitation probe including one or more optical fibers and configured to transmit light in at least the general direction;
positioning the IVUS/IVPA probe within the lumen of the blood vessel to radially probe the blood vessel;
rotating the ultrasound transducer;
probing, repeatedly while rotating, regions of the blood vessel, wherein the probing comprises:
transmitting a light pulse via the optical excitation probe to illuminate the blood vessel and induce a photoacoustic signal;
transmitting an ultrasound pulse via the ultrasound transducer to irradiate the blood vessel and induce an ultrasound echo;
receiving, using the ultrasound transducer, the photoacoustic signal and the ultrasound echo;
creating an IVUS image of the blood vessel from the received ultrasound echoes; and
creating an IVPA image of the blood vessel from the received photoacoustic signals.

21. The method for creating IVUS and IVPA images of a blood vessel according to claim 20, wherein the IVUS image and the IVPA image are co-registered due to the physical arrangement of the ultrasound transducer and the optical excitation probe.

22. The method for creating IVUS and IVPA images of a blood vessel according to claim 21, further comprising:
combining the IVUS and IVPA images to create an IVUS/IVPA image.

23. The method for creating IVUS and IVPA images of a blood vessel according to claim 22, wherein the IVUS/IVPA image is a cross-section of the blood vessel with pixel values that illustrate (i) the structure and composition of the blood vessel and (ii) the structure and composition of a plaque within the walls of the blood vessel.

24. The method for creating IVUS and IVPA images of a blood vessel according to claim 23, wherein the pixel values of the plaque illustrate lipid-rich tissues.

25. The method for creating IVUS and IVPA images of a blood vessel according to claim 23, wherein the IVUS/IVPA image facilitates a risk assessment of the plaque.

26. The method for creating IVUS and IVPA images of a blood vessel according to claim 22, further comprising:
moving the IVUS/IVPA probe along the longitudinal axis of the blood vessel while simultaneously rotating and probing to create a series of IVUS/IVPA images of contiguous segments of the blood vessel, and
reconstructing the blood vessel tomographically using the series of IVUS/IVPA images.

27. The method for creating IVUS and IVPA images of a blood vessel according to claim 26, further comprising:
identifying and mapping plaques within the walls of the blood vessel to facilitate a pathological characterization of the plaques to guide diagnosis and therapy.

28. The method for creating IVUS and IVPA images of a blood vessel according to claim 26, wherein the moving of the IVUS/IVPA along the longitudinal axis of the blood vessel comprises:
pulling the IVUS/IVPA probe through the blood vessel at a rate of less than one millimeter per second (<1 mm/sec).

29. The method for creating IVUS and IVPA images of a blood vessel according to claim 20, wherein the positioning of the probe within a blood vessel is performed in vivo or ex vivo.

30. The method for creating IVUS and IVPA images of a blood vessel according to claim 20, wherein the ultrasound transducer has a center frequency within a frequency range of 20 to 48 megahertz (MHz).

31. The method for creating IVUS and IVPA images of a blood vessel according to claim 20, wherein the wavelength of the light pulse is selected based on the absorption characteristics of the blood vessel.

32. The method for creating IVUS and IVPA images of a blood vessel according to claim 20, wherein the duration of the light pulse is selected based on the acoustic confinement criterion of the blood vessel.

33. The method for creating IVUS and IVPA images of a blood vessel according to claim 20, wherein the photoacoustic signal and the ultrasound echo are received by the ultrasound transducer at different times.

34. The method for creating IVUS and IVPA images of a blood vessel according to claim 33, wherein the different times correspond to (i) the time-of-flight of the photoacoustic signal, (ii) the time-of-flight of the ultrasound pulse plus the time-of-flight of the ultrasound echo, and (iii) a delay corresponding to the deepest structures probed by the IVUS/IVPA probe, and wherein the delay is about four microseconds (4 μs).

35. The method for creating IVUS and IVPA images of a blood vessel according to claim 20, wherein the IVUS/IVPA probe is cylindrical with a diameter less than 1.25 millimeters (mm).

36. The method for creating IVUS and IVPA images of a blood vessel according to claim 20, wherein the ultrasound transducer is rotated at a rate in the range of several revolutions per second to 30 revolutions per second.

37. The method for creating IVUS and IVPA images of a blood vessel according to claim 36, wherein the IVUS and IVPA images are created in real-time.

38. The method for creating IVUS and IVPA images of a blood vessel according to claim 36, wherein at least 250 A-lines are collected per revolution, and wherein each A-line is a mathematical representation of the photoacoustic signal and the ultrasound echo received by the ultrasound transducer.
